SEQUENCE LISTING (20 sequences)

SEQ ID NO: 1 - DNA, 26 bp, Artificial sequence
  synthetic
caaacacttattacaacccaagaaca

SEQ ID NO: 2 - DNA, 23 bp, Artificial sequence
  synthetic
tcatattatggctatgggtcagg

SEQ ID NO: 3 - DNA, 23 bp, Artificial sequence
  synthetic
aatctaccatcctccgtgaaacc

SEQ ID NO: 4 - DNA, 25 bp, Artificial sequence
  synthetic
tcagtttagctacccccaagtttaa

SEQ ID NO: 5 - DNA, 18 bp, Artificial sequence
  synthetic
cccttccccatttggtct

SEQ ID NO: 6 - DNA, 18 bp, Artificial sequence
  synthetic
tggtttcacggaggatgg

SEQ ID NO: 7 - DNA, 19 bp, Artificial sequence
  synthetic
tcctccgtgaaaccaacaa

SEQ ID NO: 8 - DNA, 18 bp, Artificial sequence
  synthetic
agcgagaagaggggcata

SEQ ID NO: 9 - DNA, 25 bp, Artificial sequence
  synthetic
gctttccacttcatcttaccattta

SEQ ID NO: 10 - DNA, 20 bp, Artificial sequence
  synthetic
tgttgggttgtttgatcctg

SEQ ID NO: 11 - DNA, 17 bp, Artificial sequence
  synthetic
cactgcctgcccagtga

SEQ ID NO: 12 - DNA, 17 bp, Artificial sequence
  synthetic
ataccgcggccgttaaa

SEQ ID NO: 13 - DNA, 18 bp, Artificial sequence
  synthetic
aacggatccacagccgta

SEQ ID NO: 14 - DNA, 18 bp, Artificial sequence
  synthetic
agtcctcgggccatgatt

SEQ ID NO: 15 - DNA, 23 bp, Artificial sequence
  synthetic
gcttaatttgactcaacacggga

SEQ ID NO: 16 - DNA, 24 bp, Artificial sequence
  synthetic
agctatcaatctgtcaatcctgtc

SEQ ID NO: 17 - DNA, 20 bp, Artificial sequence
  synthetic
acatgcagaagtcgatgcag

SEQ ID NO: 18 - DNA, 20 bp, Artificial sequence
  synthetic
cattcccctgtcgaatgtct

SEQ ID NO: 19 - DNA, 23 bp, Artificial sequence
  synthetic
tgacctcaactacatggtctaca

SEQ ID NO: 20 - DNA, 19 bp, Artificial sequence
  synthetic
cttcccattctcggccttg

(19) United States
(12) Patent Application Publication
(10) Pub. No.: US 2024/0082183 A1
(43) Pub. Date: Mar. 14, 2024

(54) METHODS FOR PREVENTING OR TREATING CONDITIONS RELATED TO T CELL MEDIATED INTESTINAL DISORDERS

(71) Applicant: The Regents of the University of Michigan, Ann Arbor, MI (US)

(72) Inventor: Pavan Reddy, Ann Arbor, MI (US)

(21) Appl. No.: 18/271,102

(22) PCT Filed: Jan. 7, 2022
(86) PCT No.: PCT/US2022/011633

§ 371 Date: Jul. 6, 2023

Related U.S. Application Data
(60) Provisional application No. 63/134,723, filed on Jan. 7, 2021.

(51) Int. Cl. A61K 31/19; A61P 37/02

(52) U.S. Cl. CPC A61K 31/19; A61P 37/02

(57) ABSTRACT

Provided herein are compositions and methods for preventing, attenuating or treating T cell mediated intestinal disorders. In particular, provided herein are methods for preventing, attenuating or treating T cell mediated intestinal disorders characterized with reduced intestinal epithelial cell (IEC) specific mitochondrial complex II component intrinsic succinate dehydrogenase A (SDHA) activity and/or expression through use of compositions comprising a therapeutic agent capable of preventing and/or hindering reduction of IEC related SDHA activity and/or expression.

### CROSS REFERENCE TO RELATED APPLICATIONS

This application claims benefit of priority to U.S. Provisional Application No. 63/134,723, filed Jan. 7, 2021, the contents of which are incorporated herein by reference in their entirety.

### STATEMENT REGARDING FEDERALLY SPONSORED RESEARCH OR DEVELOPMENT

This invention was made with government support under CA203542 awarded by the National Institutes of Health. The government has certain rights in the invention.

### FIELD OF THE INVENTION

Provided herein are compositions and methods for preventing, attenuating or treating T cell mediated intestinal disorders. In particular, provided herein are methods for preventing, attenuating or treating T cell mediated intestinal disorders characterized with reduced intestinal epithelial cell (IEC) specific mitochondrial complex II component intrinsic succinate dehydrogenase A (SDHA) activity and/or expression through use of compositions comprising a therapeutic agent capable of preventing and/or hindering reduced IEC related SDHA activity and/or expression.

### INTRODUCTION

Intestinal epithelial cells (IECs) function as critical barriers that protect from pathogen invasion. This function is disrupted by damage to IECs caused by infections or aberrant activation of immune responses^{1 2 3 4}. The non-infectious inflammatory diseases that occur in the intestinal tract such as autoimmune inflammatory bowel diseases (IBD), immune checkpoint blocker (ICB) mediated colitis, and alloimmune T cell mediated gastrointestinal graft-versus-host disease (GI-GVHD) often have similar symptoms and cause significant morbidity and mortality^{1 5 6}. Although the pathophysiology of each of these diseases is a complex interplay of the environmental and genetic factors, T cell mediated damage of IECs plays a vital role in the cause and severity of all of these conditions. Immunosuppression with corticosteroids, and anti-cytokine therapies have shown good results with adverse effects, but are still incomplete^{5 7 8}. The biology and treatments of these diseases are often understood and targeted from the immune cell and inflammation perspective, but the pathogenesis and severity from host IEC target cell perspective remain undefined.

Accordingly, improved methods for treating T cell mediated intestinal disorders are desperately needed.

The present invention addresses this urgent need.

### SUMMARY OF THE INVENTION

Emerging data in recent years have brought into focus the central role of immune cell metabolism in the regulation of intestinal inflammatory diseases. Antigen-presenting cells (APCs), including macrophages and dendritic cells, and T cells show changes in metabolic programming, including morphological alterations in mitochondria, transitioning from glycolysis dependence (pro-inflammatory macrophages and effector T cells) to oxidative phosphorylation (OXPHOS) dependence (anti-inflammatory macrophages and memory T cells)^{9 10 11} and regulate intestinal T cell mediated diseases such as GI GVHD and IBD. However, whether the metabolism of the targets of these pathogenic T cells in these diseases, the IECs, are perturbed or reprogrammed, and if so, whether this has an impact on the disease severity remains unknown. The mammalian GI tract is a relatively hypoxic region and thus, IEC metabolism is uniquely adapted to this hypoxic environment^{12 13}.

Experiments conducted during the course of developing embodiments for the present invention determined the metabolic changes in IECs when they are targeted by pathogenic T effector cells. It was found that in the context of pathogenic T cell mediated damage, the IECs demonstrated a reduction in OXPHOS and accumulated succinate as a result of decrease in SDHA, a component of mitochondrial complex II. The reduction of SDHA in IECs aggravated disease severity in multiple T cell mediated models such as the alloimmune mediated GI GVHD, autoimmune IBD and in ICB mediated colitis. Further mechanistic studies demonstrated that the disruption of mitochondrial complex II by the reduction of SDHA in the IECs was caused by the direct engagement and release of perforin dependent granzyme B by the cytotoxic T cells (CTLs) into IECs. Finally, the results were validated in human samples of GI GVHD. These results demonstrate that targeting IEC intrinsic metabolism regulates intestinal disease severity mediated by pathogenic T cells.

Accordingly, the present invention provides compositions and methods for preventing, attenuating or treating T cell mediated intestinal disorders. In particular, provided herein are methods for preventing, attenuating or treating T cell mediated intestinal disorders characterized with reduced IEC related SDHA activity and/or expression through use of compositions comprising a therapeutic agent capable of preventing and/or hindering reduced IEC related SDHA activity and/or expression.

The present invention contemplates that agents capable of preventing and/or hindering reduced IEC related SDHA activity and/or expression satisfy an unmet need for the treatment of T cell mediated intestinal disorders, either when administered as monotherapy to hinder and/or prevent reduced IED related SHDA activity and/or expression, or when administered in a temporal relationship with additional agent(s) (e.g., pharmaceutical agents known for treating T cell mediated intestinal disorders).

The embodiments of the present invention are not limited to specific certain agents capable of preventing and/or hindering reduced IEC related SHDA activity and/or expression. In some embodiments, the agent is any type or kind of moiety (e.g., small molecule, polypeptide or peptide fragment, antibody or fragment thereof, nucleic acid molecule (e.g., RNA, siRNA, microRNA, interference RNA, mRNA, replicon mRNA, RNA-analogues, and DNA), etc.) capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In some embodiments, the agent is butyrate or a compound similar to butyrate, or a pharmaceutically acceptable salt, solvate, or prodrug thereof. Such embodiments are not limited to a particular form or type of butyrate. In some embodiments, the butyrate is complexed with a metal (e.g., Na-butyrate).

Butyrate, a four-carbon short-chain fatty acid, is produced through microbial fermentation of dietary fibers in the lower intestinal tract. Butyrate has received attention for its beneficial effects on both cellular energy metabolism and intestinal homeostasis (see, Guilloteau P, et al., Nutr Res Rev 2010;23:366-84). Although it is the least abundant small chain fatty acid (SCFA) produced (˜60% acetate, 25% propionate, and 15% butyrate in humans) (see, Slavin J. Nutrients 2013;5:1417-35; Conlon MA, Bird AR. Nutrients 2014;7:17-44), butyrate is the major energy source for colonocytes (see, Chen J, et al., Curr Protein Pept Sci 2015;16:592-603; Jacobi SK, Odle J. Adv Nutr 2012;3:687-96). Butyrate modulates biological responses of host gastrointestinal health by acting as a histone deacetylase (HDAC) inhibitor and binding to several specific G protein-coupled receptors (GPCRs) (see, de Clercq NC, et al., Adv Nutr 2016;7:1080-9).

The invention also provides pharmaceutical compositions comprising such therapeutic agents capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing a T cell mediated intestinal disorders in a patient comprising a) obtaining a biological sample from the patient, wherein the biological sample comprises IECs; b) determining the presence or absence of one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs; c) administering to said patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs if the IECs are characterized as having one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing a T cell mediated intestinal disorders in a patient comprising administering to a patient suffering from or at risk of suffering from a T cell mediated intestinal disorder a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

Such embodiments are not limited to a particular type or kind of T cell mediated intestinal disorder. In some embodiments, the T cell mediated intestinal disorder is an alloimmune disorder, autoimmune disorder, and/or an iatrogenic disorder.

Autoimmune diseases are generally believed to be caused by the failure of the immune system to discriminate between antigens of foreign invading organisms (non-self) and tissues native to its own body (self). When this failure to discriminate between self and non-self occurs and the immune system reacts against self antigens, an autoimmune disorder may arise. Alloimmune diseases are referred to herein as disorders in which an immune response against or by foreign, transplanted tissue can lead to serious complications or be fatal. In the treatment of these disorders, it is desired to prevent the body from reacting against non-self antigens. Iatrogenic disorders are the result of diagnostic and therapeutic procedures undertaken on a patient.

In some embodiments, the T cell mediated intestinal disorder is an alloimmune disorder, autoimmune disorder, and/or an iatrogenic disorder selected from graft-versus-host disease (GVHD), inflammatory bowel disease (IBD) and immune checkpoint blockade (ICB) mediated colitis.

Such embodiments are not limited to a particular type or kind of patient. In some embodiments, the patient is a mammalian patient. In some embodiments, the patient is a human patient. In some embodiments, the patient is a human patient suffering from or at risk of suffering from an alloimmune disorder, an autoimmune disorder, and/or an iatrogenic disorder.

In certain embodiments, the present invention provides methods of treating, ameliorating, or preventing a reduction of IEC related SDHA activity and/or expression in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing a reduction of IEC related oxidative phosphorylation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing an increase of IEC related succinate accumulation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing an increase of IEC related accumulation of perforin dependent granzyme B related to cytotoxic T cell engagement with such IECs in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing a reduction of IEC related SDHA activity and/or expression in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing a reduction of IEC related oxidative phosphorylation in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing an increase of IEC related succinate accumulation in a biological sample comprising exposing to the biological sample a therapeutic agent capable of preventing and/or hindering one or more of reduced IEC related SDHA activity and/or expression; reduced IEC related oxidative phosphorylation; increased IEC related succinate accumulation; and increased IEC related accumulation of perform dependent granzyme B related to cytotoxic T cell engagement with such IECs.

In certain embodiments, the present invention provides methods reducing or preventing an increase of IEC related accumulation of perforin dependent granzyme B related to cytotoxic T cell engagement with such IECs in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

Such embodiments are not limited to a particular therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs. In some embodiments, the therapeutic agent is butyrate (e.g., sodium-butyrate).

Such embodiments are not limited to a particular type or kind of biological sample. In some embodiments, the biological sample is from a mammalian patient. In some embodiments, the biological sample is from a human patient. In some embodiments, the biological sample is from a human patient suffering from or at risk of suffering from an alloimmune disorder, an autoimmune disorder, and/or an iatrogenic disorder. In some embodiments, the biological sample is an in vivo, in vitro, in situ, or ex vivo biological sample. In some embodiments, the biological sample comprises IECs.

The invention also provides kits comprising one or more of the described therapeutic agents capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs and instructions for administering the compound to an animal. The kits may optionally contain other therapeutic agents.

The present invention further contemplates that agents capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs satisfy an unmet need for the treatment of multiple cancer types when administered in a temporal relationship with additional agent(s), such as other cell death-inducing or cell cycle disrupting cancer therapeutic drugs (e.g., immune checkpoint inhibitors) or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of an agent capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs and a course of an anticancer agent (e.g., immune checkpoint inhibitor) produces a greater tumor response and clinical benefit in such animals compared to those treated with the compound or anticancer drugs/radiation alone. Since the doses for all approved anticancer drugs and radiation treatments are known, the present invention contemplates the various combinations of them with the present compounds.

Such embodiments are not limited to agents capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs. In some embodiments, such an agent is a SHDA inhibitor (e.g., carboxin, thenoyltrifluoroacetone, malonate, malate, oxaloacetate).

In certain embodiments, such therapeutic agents can be used to treat, ameliorate, or prevent a cancer characterized by resistance to cancer therapies (e.g., those cancer cells which are chemoresistant, radiation resistant, hormone resistant, and the like). In some embodiments, such therapeutic agents are co-administered with an anticancer agent one or an immune checkpoint inhibitor (e.g., pembrolizumab, nivolumab, cemiplimab, atezolizumab, avelumab, durvalumab, ipilimumab), a chemotherapeutic agent, and radiation therapy. Such embodiments are not limited to particular type or kind of immune checkpoint inhibitor. In some embodiments, the immune checkpoint inhibitor is selected from a PD-1 inhibitor, PD-L1 inhibitor, CTLA-4 inhibitor, LAG3 inhibitor, TIM3 inhibitor, cd47 inhibitor, TIGIT inhibitor, and B7-H1 inhibitor. In some embodiments, the PD-1 inhibitor is selected from nivolumab, pembrolizumab, STI-A1014, pidilzumab, and cemiplimab-rwlc. In some embodiments, the PD-L1 inhibitor is selected from velumab, atezolizumab, durvalumab, and BMS-936559. In some embodiments, the CTLA-4 inhibitor is selected from ipilimumab and tremelimumab. In some embodiments, the LAG3 inhibitor is GSK2831781.

The invention also provides kits comprising one or more of the described therapeutic agents capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs and instructions for administering the compound to an animal. The kits may optionally contain other therapeutic agents (e.g., immune checkpoint inhibitors).

### BRIEF DESCRIPTION OF DRAWINGS

FIG. 1A-G: Mitochondrial respiration in IECs change after allo-HCT. B6 WT animals received 10 Gy total body irradiation and received 3×10⁶ T cells along with 5×10⁶ TCD-BM cells from either syngeneic B6 WT or allogeneic MHC-mismatched BALB/c donors. (a-c) Representative bio-energetic profiles of naïve, syngeneic or allogeneic isolated IECs on day 21 post HCT under basal conditions and following treatment with mitochondrial inhibitors (oligomycin, FCCP, rotenone). (a) oxygen consumption rate: OCR, (b) % change of OCR (oxygen consumption rate) from basal conditions, (c) basal OCR, extracellular acidification rate (ECAR) and OCR/ECAR ratio (n=5). (d) Complex I activity was evaluated in isolated mitochondria of colonic IECs from syngeneic and allogeneic recipients 7 and 21 days post HCT (n=5). (e) NADH/NAD⁺ ratio was determined in isolated IECs from naïve, syngeneic and allogeneic recipients 7 and 21 days post HCT (n=5). (f) Representative BN-PAGE image of mitochondria from IECs 21 days post HCT showing abundant mitochondrial protein complexes (n=4). (g) Representative 2D-PAGE images of mitochondria from IECs on day 21 post HCT showing reduced expression of SDHA protein (arrow, n=4). Representative plots and a graph summarizing the results of at least two independent experiments are shown. All statistical analysis by one-way ANOVA analysis with Tukey post hoc test (c), Mann-Whitney test (d, e) (mean±s.e.m.) were used to determine significance.: *P<0.05, **P<0.01, ***P<0.001.

FIG. 2A-G: Increased levels of succinate in IECs post-allo-HCT. B6 WT animals received 10 Gy total body irradiation and received 3×10⁶ T cells along with 5×10⁶ TCD-BM cells from either syngeneic B6 WT or allogeneic MHC-mismatched BALB/c donors. (a-c) Abundance of TCA metabolites on day 21 in colon tissue (a), in ileum tissue (b) and in kidney cortex (c) from syngeneic and allogeneic recipients or naïve mice (n=5). The heat maps (left) and graphical results for citrate and succinate (right) are shown. (d) Abundance from uniformly ¹³C-glucose in lactate after 4 h in syngeneic or allogeneic isolated IECs 7 and 21 days post HCT (D7 Syn: n=4, Allo: n=4, D21 Syn: n=3, Allo: n=6). (e) Carnitine and acylcarnitine levels in colon, ileum and kidney from naive, syngeneic or allogeneic recipients 21 day post HCT (n=5). (f) Isotope tracing assay from ¹³C-glucose treated mice colon 7 and 21 days post HCT (n=5). (g) Intensity of SDH enzyme activity staining from colon and ileum 21 days post HCT (n=5). Representative plots and a graph summarizing the results of at least two independent experiments are shown. All statistical analysis by one-way ANOVA analysis with Tukey post hoc test (a-c, e), unpaired t-test (d, f) or Mann-Whitney test (g) (mean±s.e.m.) were used to determine significance: *P<0.05, **P<0.01.

FIG. 3: Analysis of ¹³C incorporation from ¹³C glutamine treatment in IECs post allo-HCT. B6 WT animals received 10 Gy total body irradiation and received 3×10⁶ T cells and 5×10⁶ TCD-BM cells from either syngeneic B6 WT or allogeneic BALB/c donors. Abundance from uniformly ¹³C-glutamine of cis-aconitate, α-ketoglutarate, succinate, and malate after 4 h incubation in syngeneic or allogeneic isolated IECs 7 and 21 days post HCT (D7 Syn: n=4, Allo: n=5, D21 Syn: n=5, Allo: n=7). All statistical analysis for abundance by unpaired t-test (mean±s.e.m.): *P<0.05, **P<0.001.

FIG. 4A-C: Specific reduction of SDHA activity in colon and ileum post allo-HCT. B6 WT animals received 10 Gy total body irradiation and received 3×10⁶ (BALB/c→C57BL/B6) or 1×10⁶ (C3H.SW→C57BL/B6) T cells along with 5×10⁶ TCD-BM cells from either syngeneic or allogeneic donors. (a) Representative images of SDH enzyme activity staining in GVHD target tissues (colon, ileum, liver and skin) and non-target tissues (heart, pancreas and kidney) from naive animals or recipients 21 days post HCT (BALB/c→C57BL/B6, Scale bar: yellow 500 μm, black 200 μm). (b) Representative images of SDH enzyme activity staining in GVHD target tissues (colon, ileum) from recipients 21 days post HCT (C3H.SW→C57BL/B6, Scale bar: yellow 500 μm). (c) Integrated intensity of SDH enzyme activity staining from colon and ileum 7 days post HCT (BALB/c→C57BL/B6, n=5). Representative plots and a graph summarizing the results of at least two independent experiments are shown. All statistical analysis by Mann-Whitney test (c) (mean±s.e.m.): *P<0.05, **P<0.01.

FIG. 5A-D: Reduction of SDHA protein levels in IECs post allo-HCT. B6 WT animals received 10 Gy total body irradiation and 3×10⁶ T cells along with 5×10⁶ TCD-BM cells from either syngeneic B6 WT or allogeneic BALB/c donors. (a) Immunoblot (left) of SDHA, complex V, III, IV, SDHB, I and TOM-20 in mitochondria from IECs 21 days post HCT (n=4), and protein density of SDHA and SDHB (right) (n=4). (f) Representative BN-PAGE image of mitochondria from IECs 21 days post HCT showing abundant mitochondrial protein complexes (n=4). (b) Representative images of immunofluorescence staining of colon, ileum and heart from naive or recipients 21 days post HCT (Complex IV=green, SDHA=red, DAPI=blue, scale bar=50 μm, n=5). (c) Fluorescent intensity of SDHA in colon, ileum and heart from naive, syngeneic or allogeneic recipients 21 days post HCT (n=5). (d) Representative images of transmission electron microscopy with immune-gold staining for SDHA in mitochondria of colon from naive mice or recipients 21 days post HCT (left; scale bar 200 nm) and numbers of gold particles per mitochondrial in colon and ileum 21days post HCT (right) (50 mitochondria from 3 samples). Representative plots and a graph summarizing the results of at least two independent experiments are shown. Mann-Whitney test (a, c) or one-way ANOVA analysis with Tukey post hoc test (d) (mean±s.e.m.) were used to determine significance: *P<0.05, **P<0.01.

FIG. 6A-E: Morphological mitochondrial changes but not numbers in IECs post allo-HCT. B6 WT animals received 10 Gy total body irradiation followed by 3×10⁶ T cells along with 5×10⁶ TCD-BM cells from either syngeneic B6 WT or allogeneic BALB/c donors. (a) Immunoblot protein density quantification of SDHA and SDHB in mitochondria from IECs 7 days post HCT (n=4). (b) Representative images of immunofluorescence staining of kidney and liver from recipients 21 days post HCT (Complex IV=green, SDHA=red, DAPI=blue, scale bar=50 μm, n=5). (c) Numbers of gold particles per mitochondrial from colon and ileum of naive animals or recipients 7 days post HCT in transmission electron microscopy images with immune-gold staining of SDHA (total 50 mitochondria from 3 samples). (d) Representative images of transmission electron microscopy in mitochondria of colon from naive or recipients 21 days post HCT (left; scale bar 200 nm). Arrow indicates normal cristae and arrowhead indicates abnormal cristae (n=3). (e) Mitochondria DNA relative copy numbers of colon and ileum from syngeneic and allogeneic recipients 7 and 21 days post HCT (n=5). Representative plots and a graph summarizing the results of at least two independent experiments are shown. All statistical analysis by Mann-Whitney test (a, e) or one-way ANOVA analysis with Tukey post hoc test (c) (mean±s.e.m.): ****P<0.0001.

FIG. 7A-H: Conditioning regimen does not affect SDHA expression in IECs. (a-b) B6 WT mice received 10 Gy total body irradiation followed by 1×10⁶ T cells along with 5×10⁶ TCD-BM cells from either syngeneic B6 or allogeneic mHA-micmatched C3H.SW donors (n=5). (a) Representative images of immunofluorescence staining and (b) fluorescent intensity of SDHA in colon and ileum from recipients 28 days post HCT (scale bar=50 μm) are shown. (c-d) B6 WT mice received chemotherapy and received 1×10⁷ T cells along with 1×10⁷ TCD-BM cells from either syngeneic B6 WT or allogeneic BALB/c donors (n=5). (c) Representative images of immunofluorescence staining and (d) fluorescent intensity of SDHA in colon and ileum from recipients 21 days post HCT (scale bar=50 μm). (e-g) B6 WT mice were treated with 3% DSS or vehicle in drinking water for 7 days (n=5). (e) Representative images of immunofluorescence staining and (f) fluorescent intensity of SDHA in colon 12 days after DSS treatment (scale bar=50 μm). (g) Succinate levels in isolated IECs from colon and ileum of mice treated with 3% DSS at dayl2. (h) B6 WT mice receiving isotype control IgG or anti-CTLA-4 antibody were treated with 3% DSS in drinking water for 7 days. Time course of body weight changes after DSS administration. Representative plots and a graph summarizing the results of at least two independent experiments are shown. All statistical analysis by Mann-Whitney test (b, d, f) or unpaired t-test (g, h) (mean±s.e.m.): *P<0.05, **P<0.01.

FIG. 8A-L: T-cell mediated intestinal immunopathology and SDHA reduction in IECs. (a-c) Unirradiated B6D2F1 mice received 10×10⁷ splenocytes from syngeneic B6D2F1 or allogeneic B6 donors (n=5). (a) Representative images of immunofluorescence staining and (b) fluorescent intensity of SDHA in colon and ileum from B6D2F1 recipients 21 days post HCT (scale bar=50 μm). (c) Succinate levels in isolated IECs from colon and ileum 21 days post HCT. (d-f) B6 WT mice received 10 Gy total body irradiation without T-cell or BM cells (n=4). (d) Representative images of immunofluorescence staining and (e) fluorescent intensity of SDHA in colon and ileum from irradiated or non-irradiated (control) mice 9 days post irradiation (scale bar=50 μm). (f) Succinate levels in isolated IECs from colon and ileum 9 days post irradiation. (g-i) CD4⁺CD25⁻CD44⁻CD45RB^{hi} (naïve) T cells or CD4⁺CD25⁻CD44⁻CD45RB^{low} (non-naïve) T cells from B6 WT mice were transferred to RAG-1^{-/-} mice (n=5). (g) Representative images of immunofluorescence staining and (h) fluorescent intensity of SDHA in colon and ileum 8 weeks after induction of colitis (scale bar=50 μm). (i) Succinate levels in isolated IECs from colon and ileum at 8 weeks after T cell transfer. (j-l) B6 WT mice receiving isotype control IgG or anti-CTLA-4 antibody were treated with 3% DSS in drinking water for 7 days (n=5). (j) Representative images of immunofluorescence staining and (k) fluorescent intensity of SDHA in colon from mice receiving IgG or CTLA-4 antibody 12 days after DSS treatment (scale bar=50 μm). (l) Succinate levels in isolated IECs from colon and ileum 12 days after 3% DSS administration. Representative plots and a graph summarizing the results of at least two independent experiments are shown. All statistical analysis by Mann-Whitney test (b, e, h, k) or unpaired t-test (c, f, i, l) (mean±s.e.m.) were used to determine significance: *P<0.05, **P<0.01.

FIG. 9A-H: Functional relevance of SDHA reduction in IECs. (a-b) Primary colonic epithelial cells (PCECs) were treated with DMSO, malonate or itaconate for 6 hours (n=5). (a) Representative FACS plots and (b) Annexin-V positive cells are shown. (c) PCECs were treated with 4-Octyl itaconate (0.1 mM) for 6 hours. Annexin-V positive cells and MitoSox positive cells are shown (n=5) (d) B6 WT animals received 10 Gy total body irradiation and received 3×10⁶ T cells along with 5×10⁶ TCD-BM cells from either syngeneic B6 WT or allogeneic BALB/c donors. ATP production in isolated mitochondria of colon from naïve, syngeneic and allogeneic recipients 21 days post HCT (n=3). Mitochondria were treated with vehicle, malonate or vehicle without ADP. (e) Isolated splenic T cells from either syngeneic B6 WT or allogeneic BALB/c animals were cultured with irradiated splenocytes derived from B6 WT animals for 96 hours, and CD8⁺T cells were purified as effector cells. PCECs from B6 WT animals were treated with vehicle, malonate or itaconate and used as targets for effector T-cells. ⁵¹Cr-release assay using effector cells against PCECs was analyzed (n=3 biological replicates and n=2 technical replicates). (f) Isolated splenic T cells were purified as effector cells as shown in (e). PCECs were treated with vehicle or 4-Octyl itaconate (0.2 mM) for 2 hours and used as targets for effector T-cells. AnnexinV positive cell of Green CMFDA Dye positive PCECs was analyzed (n=4 biological replicates and n=2 technical replicates). (g) PCECs pre-treated with NAD (1 mM) for 2 hours were treated with perforin and Granzyme B for 4 hours, and were analyzed for the apoptosis and Mitosox levels (Medium: n=15, Vehicle: n=18, NAD: n=18). (h) Isolated splenic T cells were purified as effector cells as shown in (e). PCECs were treated with vehicle or NAD for 2 hours and used as targets for effector T-cells. Apoptosis was analyzed (n=3 biological replicates and n=2 technical replicates). Representative plots and a graph summarizing the results of at least two independent experiments are shown. All statistical analysis by one-way ANOVA analysis with Tukey post hoc test (b, d, f, g, h) or unpaired t-test (c, e) (mean±s.e.m.) were used to determine significance: *P<0.05, **P<0.01, ***P<0.001.

FIG. 10A-K: SDH inhibition causes ROS accumulation in IECs. (a-e) Primary colonic epithelial cells (PCECs) were treated with DMSO, malonate or itaconate for 6 hours (n=5). (a-b) Cytoplasmic ROS measured by CellROX staining. (a) Representative images and (b) CellROX positive cells are shown. (c-d) Mitochondria ROS measured by MitoSOX staining. (c) Representative images and (d) Cell SOX positive cells are shown. (e) PCECs were treated with DMSO or atpeninA5 for 4 hours. Apoptosis (left), CellROX (middle) and MitoSOX (right) levels were determined (n=5). (f-h) B6 WT animals received 10 Gy total body irradiation followed by 3×10⁶ T cells along with 5×10⁶ TCD-BM cells from either syngeneic B6 WT or allogeneic BALB/c donors (n=5). (f) CellROX and MitoSOX levels in isolated colon and ileum IECs from syngeneic and allogeneic recipients 7 and 21 days post HCT were analyzed. (g) Representative plots and (h) levels of mitochondrial membrane potential (wm) in isolated colon and ileum IECs from syngeneic and allogeneic recipients 7 and 21 days post HCT were analyzed. (i) PCECs pre-treated with Tiron, Trolox, and GDF15 for 2 hours were treated with perforin and Granzyme B for 4 hours. PCECs were analyzed for the apoptosis and Mitosox levels (n=7). (j, k) B6 WT animals received 10 Gy total body irradiation followed by 3×10⁶ T cells along with 5×10⁶ TCD-BM cells from either syngeneic B6 WT or allogeneic BALB/c donors. (j) Immunoblot of SUCNR1 in peritoneal macrophage (MF) from naïve B6, IECs from naïve, syngeneic and allogeneic recipients 7 days post HCT. (k) Transcriptome analysis of IECs from syngeneic and allogeneic recipients 7 days post HCT. The mRNA expression of HIFla and cytochromeB (n=3). Representative plots and a graph summarizing the results of at least two independent experiments are shown. All statistical analysis by one-way ANOVA analysis with Tukey post hoc test (b, d, e, i) or unpaired t-test (f, h) (mean±s.e.m.), The p-values of the transcriptome analysis (k) obtained from DESeq-analysis. Differential platelet mRNA expression was calculated relative to the control group. *P<0.05, **P<0.01, ***P<0.001.

FIG. 11A-I: Chemical inhibition and genetic ablation of SDHA in IECs regulate the severity of GVHD. (d) Representative images of SDH enzyme staining of colon from naïve B6 WT 12 hours after vehicle, malonate (5 g kg⁻¹) or itaconate (2.5 g kg⁻¹) treatment. (Scale bar 100 μm, n=2). (e-f) B6 WT or Sdhaf1^{-/-} animals received 10 Gy total body irradiation followed by 3×10⁶ T cells along with 5×10⁶ TCD-BM cells from either syngeneic B6 WT or allogeneic BALB/c donors. (e) Survival of B6 WT recipients treated with vehicle or atpeninA5 (9 μg kg⁻¹) every other day post HCT (n=8). (f) B6 WT recipients treated with vehicle, malonate (5 g kg⁻¹) or itaconate (2.5 g kg⁻¹) every other day post HCT. Pathological GVHD scores in liver, skin and lung 7 days post HCT (n=6). (g) Pathological GVHD scores in liver, skin and lung 7 days post HCT from B6 WT and Sdhaf1^{-/-} recipients (n=6). (h) B6 WT and Sdhaf1^{-/-} animals were treated with 2.5 DSS in drinking water for 7 days. Time course of body weight changes after DSS administration (n=6). (i) Scheme illustrating the strategy used to generate Sdha floxed and excised alleles in IECs. (j) Expression of Sdha mRNA in CD326⁺ isolated IECs from six-week-old Sdha^{Δ/IEC} relative to Sdha^{fl/IEC} mice (n=4). Sdha expression was normalized to GAPDH expression. (k) Immunoblot of SDHA and TOM-20 in mitochondria of IECs from six-week-old Sdha^{Δ/IEC} and Sdha^{fl/IEC} mice. (l) Representative images of immunofluorescence staining of colon, ileum, liver, skin and heart from six-week-old Sdha^{Δ/IEC} and Sdha^{fl/IEC} mice (Complex IV=green, SDHA=red, DAPI=blue, scale bar=50 μm, n=4). Representative plots and a graph summarizing the results of at least two independent experiments are shown. All statistical analysis by log-rank test (b), Mann-Whitney test (d, e, g) or Kruskal-Wallis analysis with Dunn's post hoc test (b) (mean±s.e.m.), *P<0.05, **P<0.01, ***P<0.001.

FIG. 12A-O: SDHA in IECs regulates the severity of GVHD. B6 WT, Sdhaf1^{-/-}, Sdha^{Δ/IEC} or Sdha^{fl/IEC} animals received 10 Gy total body irradiation followed by 3×10⁶ T cells and 5×10⁶ TCD-BM cells from either syngeneic B6 WT or allogeneic BALB/c donors. (a-c) B6 WT recipients were treated with vehicle, malonate (5 g kg⁻¹) or itaconate (2.5 g kg⁻¹) every other day post HCT. Survival (a), clinical GVHD severity (b) and pathological GVHD scores in ileum and colon 7 days post HCT (c) (n=6). (d-f) B6 WT and Sdhaf1^{-/-} mice received HCT (n=6). Survival (d), clinical GVHD severity (e) and pathological GVHD scores in ileum and colon 7 days post HCT (f). (g) Representative images of immunofluorescence staining and (h) fluorescent intensity of colon and ileum from B6WT and Sdhaf1^{-/-} mice 7 days post allo-HCT (Complex IV=green, SDHA=red, DAPI=blue, scale bar=50 μm, n=6). (i) Succinate levels in isolated IECs from colon and ileum of B6WT and Sdhaf1^{-/-} mice at day 7 post allo-HCT (n=6). (j) Survival of chimeric [B6 Ly5.2→B6 WT] and [B6 Ly5.2→Sdhaf1^{-/-}] animals receiving 9 Gy TBI followed by 3×10⁶ T cells and 5×10⁶ TCD-BM cells from syngeneic B6WT or allogeneic BALB/c donors. (k) Survival and clinical GVHD severity (l) of Sdha^{Δ/IEC} and Sdha^{fl/IEC} mice post HCT (syngeneic Sdha^{Δ/IEC} and Sdha^{fl/IEC}:n=6, allogeneic Sdha^{Δ/IEC}:n=8, allogeneic Sdha^{fl/IEC}:n=10). (m-o) B6 WT and Irg1^{-/-} animals received 10 Gy total body irradiation followed by 3×10⁶ T cells and 5×10⁶ TCD-BM cells from syngeneic B6 WT or allogeneic BALB/c donors. (m) Images (left) and fluorescent intensity of immunofluorescence staining (right) of colon from B6 WT and Irg1^{-/-} mice 21 days after post allo-HCT (Complex IV=green, SDHA=red, DAPI=blue, scale bar=50 μm, n=3). Clinical GVHD severity (n) and pathological GVHD score of ileum and colon (o) from WT B6 and Irg1^{-/-} mice post HCT (Clinical GVHD severity, syngeneic B6 WT: n=8, allogeneic B6 WT: n=15, allogeneic Irg1^{-/-}:n=15, pathological GVHD score, n=3). All statistical analysis by log-rank test (a, d, j, k), Mann-Whitney test (b, e, f, h, l), Kruskal-Wallis analysis with Dunn's post hoc test (c, n) or unpaired t-test (i, m, o) (mean±s.e.m.): *P<0.05, **P<0.01, ***P<0.001.

FIG. 13A-C: Transcriptome analysis of IECs after allo-HCT. (a-c) B6 WT animals received 10 Gy total body irradiation followed by injection of 3×10⁶ T cells along with 5×10⁶ TCD-BM cells from either syngeneic B6 WT or allogeneic BALB/c donors. Transcriptome analysis of IECs from syngeneic and allogeneic recipients 7 days post HCT. Heatmap of OXPHOS pathway gene (a) and TCA cycle related gene (b). (c) The mRNA expression of SDHA, SDHB and SDHC in IECs from syngeneic and allogeneic recipients (n=3). The p-values of the transcriptome analysis (c) obtained from DESeq-analysis

FIG. 14A-K: Granzyme B dependent abrogation of SDHA levels in IECs. (a-b) (a) Isolated splenic T cells from either B6 WT or BALB/c mice were cultured with irradiated splenocytes derived from B6 WT for 96 hours and CD8⁺T cells were purified as effector cells. 51Cr-release assay using effector cells against PCECs was determined in direct or indirect co-culture with T-cells (using transwell membrane) or supernatant treatment from effector cell culture after 12 hours treatment (n=5 biological replicates and n=2 technical replicates). (b) Representative images of immunofluorescence staining of CD326⁺PCECs from T cell treatment as shown in (a) (Complex IV=green, SDHA=red, DAPI=blue, scale bar=5 μm, n=5). (c) Immunoblot(left) of SDHA, Complex V, IV, III, SDHB, I and TOM-20 in PCECs treated with perforin and Granzyme B for 4 hours. (d) Normal SDHA protein and mutated SDHA protein were treated perforin and Granzyme B for 6 hours. Immunoblot image and ratio of medium/GranzymeB+SDHA were shown (n=6). (e-g) BALB/c WT animals received 8 Gy total body irradiation and received 1×10⁶ T cells and 5×10⁶ TCD-BM cells from B6 WT or Prf^{-/-} donors. (e) Representative images of immunofluorescence staining and (f) fluorescent intensity of colon and ileum from BALB/c mice that received B6 WT or Prf^{-/-} donor cells 21 days post allo-HCT (Complex IV=green, SDHA=red, DAPI=blue, scale bar=50 μm, n=5). (g) Succinate levels in isolated IECs from colon and ileum of BALB/c mice received B6 WT or Prf^{-/-} donor cells 21 days post allo-HCT (n=5). (h-k) Naïve T cells from B6 WT or Prf^{-/-} mice or non-naïve T-cells from B6 WT mice were transferred to RAG-1^{-/-} mice. (h) Time course of body weight changes after T-cell transfer (n=5). (h) Representative images of immunofluorescence staining and (i) fluorescent intensity of colon and ileum 8 weeks after induction of colitis (scale bar=50 μm, n=5). (j) Succinate levels in isolated IECs from colon and ileum of RAG-1^{-/-} mice 8 weeks after T cell transfer (n=5). Representative plots and a graph summarizing the results of at least two independent experiments are shown. All statistical analysis by unpaired t-test (a, g), Mann-Whitney test (c, d, f, j) and one-way ANOVA analysis with Tukey post hoc test (h, k) (mean±s.e.m.) were used to determine significance: *P<0.05, **P<0.01, ***P<0.001.

FIG. 15A-K: Granzyme B cleaves SDHA in mitochondria from IECs. (a) PCECs treated with perforin and Granzyme B for 4 hours were analyzed for AnnexinV positive cells, CellROX, and MitoSOX levels (n=4). (b) Amino acid sequences of SDHA, SDHB and mutated SDHA. A possible target by Granzyme B was shown in gray and. (c) Isolated mitochondria were incubated with Granzyme B in indicated time (ON; overnight). Immunoblot image of full length or cleaved SDHA and cleaved/full length SDHA ratio were shown (n=3, each). (d) His-Tagged recombinant SDHA and SDHB proteins were treated with Granzyme B for 90 minutes. Immunoblot image and ratio of cleaved/full length SDHA/SDHB were shown (n=3, each). (e) PCECs were treated with perforin, Granzyme B, and Serpin 3A for 6 hours. Immunoblot image of SDHA and the protein density of SDHA were shown (n=4). (f) BALB/c WT animals received 8 Gy total body irradiation followed by 1×10⁶ T cells along with 5×10⁶ TCD-BM cells from B6 WT or Prf^{-/-} donors. Succinate levels in isolated IECs from colon and ileum of BALB/c mice that received B6 WT or Prf^{-/-} donor cells 7 days post allo-HCT (n=5). (g) BALB/c WT animals received 8 Gy total body irradiation followed by 1×10⁶ T cells along with 5×10⁶ TCD-BM cells from B6 WT or FasL^{-/-} donors. Representative images of immunofluorescence staining and fluorescent intensity of colon from BALB/c mice that received B6 WT or FasL^{-/-} donor cells 21 days post allo-HCT (Complex IV=green, SDHA=red, DAPI=blue, scale bar=50 μm, n=5). (h-k) Naïve T cells from B6 WT or Prf^{-/-} mice or non-naïve T-cells from B6 WT mice were transferred to RAG-1^{-/-} mice (n=5). (h) Representative images of immunofluorescence staining of colon 8 weeks after induction of colitis (Granzyme B=red, DAPI=blue, scale bar=50 μm) and (i) Granzyme B positive cell numbers in colon. (j) CellROX and (k) MitoSOX levels were determined in colonic IECs 8 weeks after induction of colitis (n=5). All statistical analysis by one-way ANOVA analysis with Tukey post hoc test (a, i, j, k), Mann-Whitney test (e), or unpaired t-test (d, f, g) (mean±s.e.m.): *P<0.05, **P<0.01, ***P<0.001.

FIG. 16A-B: Clinical correlation of SDHA levels in IECs from human clinical samples. (a-b) Representative images of immunofluorescence staining of colonic biopsy samples from patients clinically suspected to have lower GI GVHD after allo-HCT (a, scale bar=50 μm, GVHD: n=15, non-GVHD: n=16) and (b) Integrated fluorescent intensity of SDHA levels. All statistical analysis by unpaired t-test (b) (mean±s.e.m.) were used to determine significance: ***P<0.001.

FIG. 17: SDH expression in IECs from GVHD and non-GVHD patients. Other representative images of immunofluorescence staining of colonic biopsy samples from patients suspected as having clinical GVHD after HCT not shown in FIG. 8 (scale bar=50 μm).

FIG. 18A-G shows the ability of butyrate to reduce IEC related SDHA activity and/or expression; reduce IEC related oxidative phosphorylation; increase IEC related succinate accumulation; and increase IEC related accumulation of perforin dependent granzyme B related to cytotoxic T cell engagement with such IECs.

### DETAILED DESCRIPTION OF THE INVENTION

Intestinal epithelial cells (IECs) function as critical barriers that protect from pathogen invasion. This function is disrupted by damage to IECs caused by infections or aberrant activation of immune responses^{1 2 3 4}. The non-infectious inflammatory diseases that occur in the intestinal tract such as autoimmune inflammatory bowel diseases (IBD), immune checkpoint blocker (ICB) mediated colitis, and alloimmune T cell mediated gastrointestinal graft-versus-host disease (GI-GVHD) often have similar symptoms and cause significant morbidity and mortality^{1 5 6}. Although the pathophysiology of each of these diseases is a complex interplay of the environmental and genetic factors, T cell mediated damage of IECs plays a vital role in the cause and severity of all of these conditions. Immunosuppression with corticosteroids, and anti-cytokine therapies have shown good results with adverse effects, but are still incomplete^{5 7 8}. The biology and treatments of these diseases are often understood and targeted from the immune cell and inflammation perspective, but the pathogenesis and severity from host IEC target cell perspective remain undefined.

Emerging data in recent years have brought into focus the central role of immune cell metabolism in the regulation of intestinal inflammatory diseases. Antigen-presenting cells (APCs), including macrophages and dendritic cells, and T cells show changes in metabolic programming, including morphological alterations in mitochondria, transitioning from glycolysis dependence (pro-inflammatory macrophages and effector T cells) to oxidative phosphorylation (OXPHOS) dependence (anti-inflammatory macrophages and memory T cells)^{9 10 11} and regulate intestinal T cell mediated diseases such as GI GVHD and IBD. However, whether the metabolism of the targets of these pathogenic T cells in these diseases, the IECs, are perturbed or reprogrammed, and if so, whether this has an impact on the disease severity remains unknown. The mammalian GI tract is a relatively hypoxic region and thus, IEC metabolism is uniquely adapted to this hypoxic environment^{12 13}.

Experiments conducted during the course of developing embodiments for the present invention aimed to determine the metabolic changes in IECs when they are targeted by pathogenic T effector cells. It was found that in the context of pathogenic T cell mediated damage, the IECs demonstrated a reduction in OXPHOS and accumulated succinate as a result of decrease in SDHA, a component of mitochondrial complex II. The reduction of SDHA in IECs aggravated disease severity in multiple T cell mediated models such as the alloimmune mediated GI GVHD, autoinimune IBD and in ICB mediated colitis. Further mechanistic studies demonstrated that the disruption of mitochondrial complex II by the reduction of SDHA in the IECs was caused by the direct engagement and release of perforin dependent granzyme B by the cytotoxic T cells (CTLs) into IECs. Finally, the results were validated in human samples of GI GVHD. These results demonstrate that targeting IEC intrinsic metabolism regulates intestinal disease severity mediated by pathogenic T cells.

Accordingly, the present invention provides compositions and methods for preventing, attenuating or treating T cell mediated intestinal disorders. In particular, provided herein are methods for preventing, attenuating or treating T cell mediated intestinal disorders characterized with reduced IEC related SDHA activity and/or expression through use of compositions comprising a therapeutic agent capable of preventing and/or hindering reduced IEC related SDHA activity and/or expression.

In certain embodiments, the present invention provides methods of treating, ameliorating, or preventing a T cell mediated intestinal disorders in a patient comprising a) obtaining a biological sample from the patient, wherein the biological sample comprises IECs; b) determining the presence or absence of one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs; c) administering to said patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs if the IECs are characterized as having one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing a T cell mediated intestinal disorders in a patient comprising administering to a patient suffering from or at risk of suffering from a T cell mediated intestinal disorder a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

Such embodiments are not limited to a particular type or kind of T cell mediated intestinal disorder. In some embodiments, the T cell mediated intestinal disorder is an alloimmune disorder, autoimmune disorder, and/or an iatrogenic disorder.

Autoimmune diseases are generally believed to be caused by the failure of the immune system to discriminate between antigens of foreign invading organisms (non-self) and tissues native to its own body (self). When this failure to discriminate between self and non-self occurs and the immune system reacts against self antigens, an autoimmune disorder may arise. Alloimmune diseases are referred to herein as disorders in which an immune response against or by foreign, transplanted tissue can lead to serious complications or be fatal. In the treatment of these disorders, it is desired to prevent the body from reacting against non-self antigens. Iatrogenic disorders are the result of diagnostic and therapeutic procedures undertaken on a patient.

In some embodiments, the T cell mediated intestinal disorder is an alloimmune disorder, autoimmune disorder, and/or an iatrogenic disorder selected from graft-versus-host disease (GVHD), inflammatory bowel disease (IBD) and immune checkpoint blockade (ICB) mediated colitis.

Such embodiments are not limited to a particular type or kind of patient. In some embodiments, the patient is a mammalian patient. In some embodiments, the patient is a human patient. In some embodiments, the patient is a human patient suffering from or at risk of suffering from an alloimmune disorder, an autoimmune disorder, and/or an iatrogenic disorder.

In certain embodiments, the present invention provides methods of treating, ameliorating, or preventing a reduction of IEC related SDHA activity and/or expression in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing a reduction of IEC related oxidative phosphorylation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing an increase of IEC related succinate accumulation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods of treating, ameliorating, or preventing an increase of IEC related accumulation of perforin dependent granzyme B related to cytotoxic T cell engagement with such IECs in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing a reduction of IEC related SDHA activity and/or expression in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing a reduction of IEC related oxidative phosphorylation in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing an increase of IEC related succinate accumulation in a biological sample comprising exposing to the biological sample a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

In certain embodiments, the present invention provides methods reducing or preventing an increase of IEC related accumulation of perforin dependent granzyme B related to cytotoxic T cell engagement with such IECs in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

Such embodiments are not limited to a particular therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs. In some embodiments, the therapeutic agent is butyrate (e.g., sodium-butyrate).

Such embodiments are not limited to a particular type or kind of biological sample. In some embodiments, the biological sample is from a mammalian patient. In some embodiments, the biological sample is from a human patient. In some embodiments, the biological sample is from a human patient suffering from or at risk of suffering from an alloimmune disorder, an autoimmune disorder, and/or an iatrogenic disorder. In some embodiments, the biological sample is an in vivo, in vitro, in situ, or ex vivo biological sample. In some embodiments, the biological sample comprises IECs.

The present invention further contemplates that agents capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs satisfy an unmet need for the treatment of multiple cancer types when administered in a temporal relationship with additional agent(s), such as other cell death-inducing or cell cycle disrupting cancer therapeutic drugs (e.g., immune checkpoint inhibitors) or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of an agent capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs and a course of an anticancer agent (e.g., immune checkpoint inhibitor) produces a greater tumor response and clinical benefit in such animals compared to those treated with the compound or anticancer drugs/radiation alone. Since the doses for all approved anticancer drugs and radiation treatments are known, the present invention contemplates the various combinations of them with the present compounds.

Such embodiments are not limited to agents capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs. In some embodiments, such an agent is a SHDA inhibitor (e.g., carboxin, thenoyltrifluoroacetone, malonate, malate, oxaloacetate).

In certain embodiments, such therapeutic agents can be used to treat, ameliorate, or prevent a cancer characterized by resistance to cancer therapies (e.g., those cancer cells which are chemoresistant, radiation resistant, hormone resistant, and the like). In some embodiments, such therapeutic agents are co-administered with an anticancer agent one or an immune checkpoint inhibitor (e.g., pembrolizumab, nivolumab, cemiplimab, atezolizumab, avelumab, durvalumab, ipilimumab), a chemotherapeutic agent, and radiation therapy.

Such embodiments are not limited to particular type or kind of immune checkpoint inhibitor. In some embodiments, the immune checkpoint inhibitor is a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

In some embodiments, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, pidilzumab, and cemiplimab-rwlc. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See, e.g., Unanue, E.R., PNAS 110:10886-87 (2013).

In another embodiment, the immune checkpoint inhibitor is a TIGIT inhibitor. See, e.g., Harjunpää 1 and Guillerey, Clin Exp Immunol 200:108-119 (2019).

In another embodiment, the immune checkpoint inhibitor is a polypeptide that binds to and blocks PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

In another embodiment, the immune checkpoint inhibitor is a compound with peptide moieties that inhibit PD-1 signaling. Examples of such compounds are disclosed in U.S. Pat. No. 8,907,053.

In another embodiment, the immune checkpoint inhibitor is an inhibitor of certain metabolic enzymes, such as indoleamine 2,3 dioxygenase (IDO), which is expressed by infiltrating myeloid cells and tumor cells, and isocitrate dehydrogenase (IDH), which is mutated in leukemia cells. Mutants of the IDH enzyme lead to increased levels of 2-hydroxyglutarate (2-HG), which prevent myeloid differentiation. Stein et al., Blood 130:722-31 (2017); Wouters, Blood 130:693-94 (2017). Particular mutant IDH blocking agents include, but are not limited to, ivosidenib and enasidenib mesylate. Dalle and DiNardo, Ther Adv Hematol 9(7):163-73 (2018); Nassereddine et al., Onco Targets Ther 12:303-08 (2018). The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In another embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In another embodiment, the immune checkpoint inhibitor is pembrolizumab, nivolumab, cemiplimab, atezolizumab, avelumab, durvalumab, or ipilimumab.

In another embodiment, the immune checkpoint inhibitor is nivolumab.

Compositions within the scope of this invention include all compositions wherein the described agents capable of 1) preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs, or 2) one or more of increasing IEC related SDHA activity and/or expression; increasing IEC related oxidative phosphorylation; decreasing IEC related succinate accumulation; and decreasing IEC related accumulation of perforin dependent granzyme B related to cytotoxic T cell engagement with such IECs are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the described agents may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I. Analysis of Mitochondrial Respiration in IECs

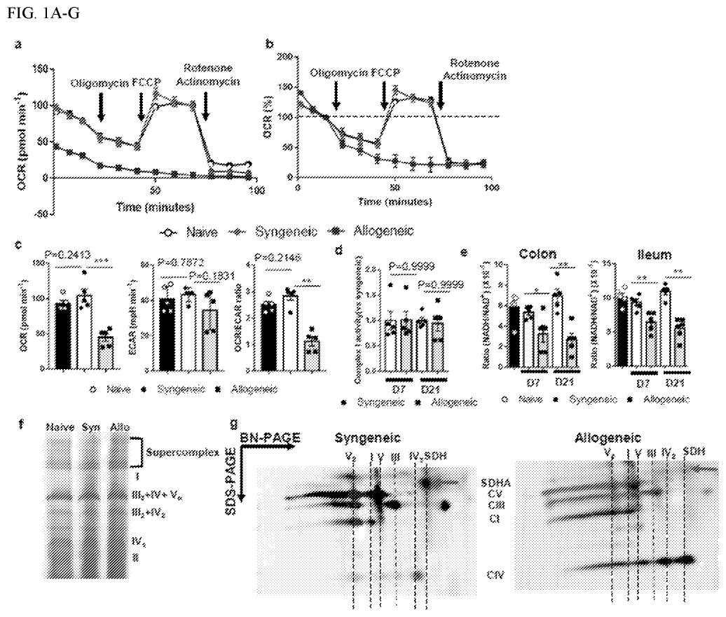

We (the applicants) explored the impact of immune mediated attack on the bioenergetics of intestinal target cells, IECs. B6 WT animals were lethally irradiated and transplanted with bone marrow (BM) and splenic T cells from either syngeneic B6 or allogeneic BALB/c donors. CD326⁺IECs from un-transplanted naïve B6 animals, the syngeneic, and allogeneic animals were harvested 21 d after hematopoietic stem cell transplantation (post HCT) and their bio-energetic profiles were analyzed with Seahorse XF ¹⁴. Compared with IECs from naïve and syngeneic animals, IECs from allogeneic animals demonstrated significantly lower oxygen consumption rates (OCR), but similar extracellular acidification rates (ECAR, an indicator of glycolysis), which dramatically reduced the OCR/ECAR ratio (FIG. 1a-c). IECs from allogeneic animals also did not respond to treatment with carbonyl cyanide-p-trifluoromethoxyphenyihydrazone (FCCP), a mitochondrial uncoupler, when compared to IECs from syngeneic animals (FIG. 1b) demonstrating that the mitochondrial electron transport chain (ETC) functions in IECs from allogeneic mice was reduced. ETC reactions begin at both complex II and complex II. Therefore, to analyze complex I function in IECs, we isolated mitochondria from recipients 21 d post HCT and analyzed their ability to oxidize NADH to NAD⁺. There was no difference in the mitochondrial complex I functions in the IECs harvested from syngeneic and allogeneic recipients (FIG. 1d). Although mitochondrial complex I function was not altered, the net NADH/NAD⁺ ratio was lower in IECs from allogeneic animals than in IECs from syngeneic animals 7 d and 21 d post HCT (FIG. 1e) suggesting that complex II function may be the cause for altered ETC in IECs after allo-HCT. Therefore, we next assessed for mitochondrial complex II and the other super complex levels, with Blue Native Polyacrylamide Gel Electrophoresis (BN-PAGE). Analysis of IECs from naïve, syngeneic, and allogeneic recipients post HCT with BN-PAGE showed alteration of mitochondrial complex II but did not show changes in other mitochondrial supercomplex levels (FIGS. 1f and g). These results demonstrated that alto-HCT induced changes in mitochondria ETC and caused disruption of complex II in IECs.

### Example II. TCA Cycle Metabolite Profiling in IECs

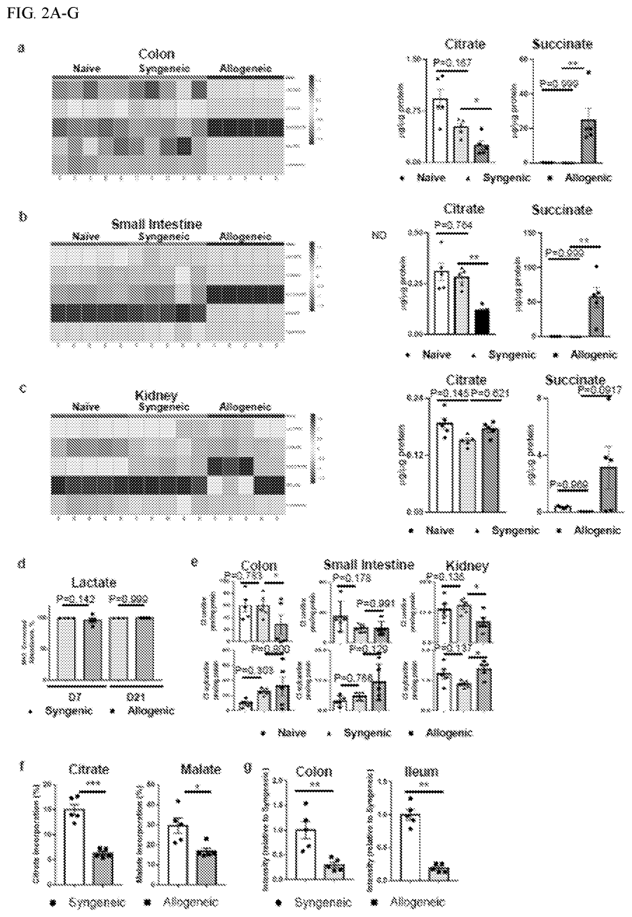

Because oxidative phosphorylation (OXPHOS) and mitochondrial respiration are linked, and complex II is critical for both processes, we hypothesized that alterations in the bioenergetics of the IECs would also result in alteration of the metabolites from oxidative phosphorylation. We focused on the TCA cycle, a critical metabolic hub for generation of adenosine triphosphate (ATP) energy from the oxidation of carbohydrates, fats, and proteins. The IECs were harvested twenty-one days post-HCT as above, and were profiled for all of the TCA cycle-related metabolites using Liquid chromatography mass spectrometry (LC/MS). The kidneys from the same animals were harvested and subjected to similar analysis as controls for non-GVHD target organs (kidneys) from the animals. The IECs from the naïve and syngeneic animals were not significantly different for any of the metabolites measured; however, the levels of succinate were significantly elevated, whereas the levels of malate, and fumarate were decreased in the IECs harvested from the colons of allogeneic recipients when compared with naïve and syngeneic recipients (FIG. 2a). Similarly, significant increase in accumulation of succinate was observed in the IECs from small intestine of allogeneic recipients (FIG. 2b). By contrast, in the non-GVHD control target organ, kidneys, there were no differences in TCA metabolite levels between the naïve, syngeneic and allogeneic recipients (FIG. 2c). Lactate levels, an indicator of glycolysis, were next measured to assess for potential shift of energy production from OXPHOS to glycolysis. The levels of lactate were similar in the IECs harvested from naïve, syngeneic, and allogeneic recipients, suggesting a lack of impact on glycolysis. To further confirm lack of shift from OXPHOS to glycolysis, IECs isolated from syngeneic or allogeneic recipients 7 d and 21 d post HCT were cultured in ¹²C-glucose or ¹³C-glucose for 4 h and analyzed for incorporation of ¹³C into glycolysis. There were no differences in incorporation of ¹³C into lactate in syngeneic and allogeneic IECs (FIG. 2d). These data demonstrate that succinate levels were significantly elevated in IECs post allo-HCT without a concomitant change in the levels of the lactate.

### Example III. Mechanisms for Increased Levels of Succinate in the IECs

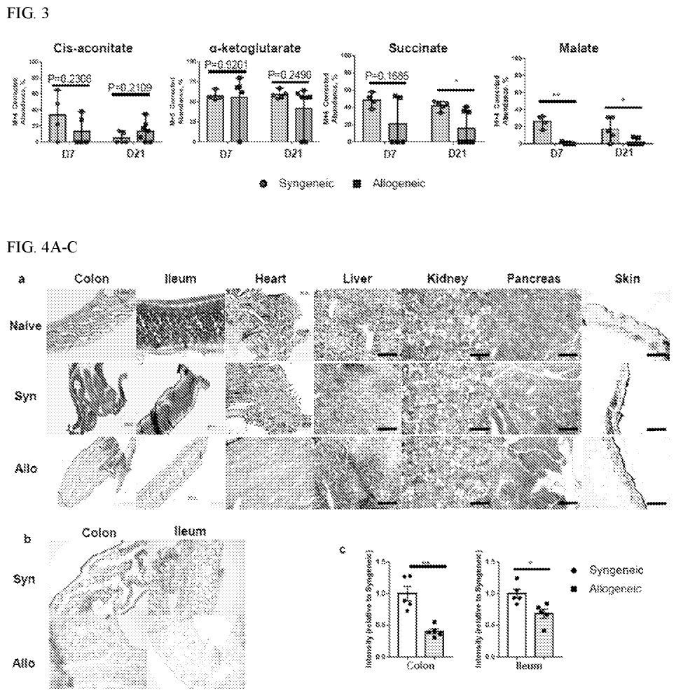

We next explored the mechanisms for succinate accumulation in the IECs from allogeneic recipients. IECs utilize fatty acids supplied from beta-oxidation as their key sources of energy^{15,16}. Therefore, high levels of succinate could be the result of enhanced beta oxidation or anaplerosis. We investigated carnitine levels in each organ as a surrogate for beta-oxidation, and no differences were observed among the groups in the IECs and also in the various organs (FIG. 2e). We next performed isotope tracing assay with LC/MS to assess incorporation of ¹³C-glucose into IEC glucose pools after transplantation. We treated syngeneic and allogeneic transplant recipients with an oral bolus of ¹³C-glucose or ¹²C-glucose (the control) 7 d after the bone marrow transplant (BMT). The IECs from the syngeneic and allogeneic animals were harvested 4 h later and analyzed for incorporation of ¹³C. There was significantly less incorporation of ¹³C into malate in the IECs harvested from allogeneic animals suggesting a reduction in TCA cycle activity (FIG. 2f). To investigate whether anaplerosis from glutamine contributed to succinate accumulation in the TCA cycle we once again performed MFA with LC/MS to measure ¹³C-glutamine in the IECs isolated from various above groups. To this end, IECs were harvested from syngeneic and allogeneic transplant recipients 7 and 21 days after HCT, treated with ¹³C-glutamine or ¹²C-glutamine for 4 h, and then analyzed for incorporation of ¹³C. We detected similar levels of ¹³C incorporation into both alpha-ketoglutarate and succinate in syngeneic and allogeneic IECs (FIG. 3) suggesting that enhanced anaplerosis from glutamine did not contribute to greater levels of succinate in the allo-IECs.

The above data suggested that increased levels of succinate was not from an increase in influx, but likely because complex II was affected. We therefore hypothesized that high level of succinate is a function of reduction in the enzyme succinate dehydrogenase A (SDHA), an enzyme that catalyzes the oxidation of succinate to fumarate, and generates FADH₂, which carries electrons and reduces ubiquinone (Q) to ubiquinol (QH₂) in ETC. We evaluated SDHA activity in the IECs, GVHD target and non-target organs after transplantation. We stained cryosections of GVHD target organs (colon, ileum, liver, and skin) and non-GVHD target organs (kidney, pancreas, and heart) as controls from the recipients on days 7 and 21 post HCT to assess for SDHA enzymatic activity (FIG. 4a,b). SDH activity was preserved in the liver, skin, kidney, pancreas, and heart in naïve and post HCT tissues, whereas SDH activity was significantly reduced in the colon and the ileum, on both days 7 and 21 post allo-HCT (FIG. 2g and FIG. 4c). These data demonstrate that the accumulation of succinate is secondary to reduction in the activity of mitochondrial complex II component, SDHA enzyme, in the IECs from allogeneic recipients.

### Example IV. Validation of Reduction in SDHA Component of Mitochondrial Complex II

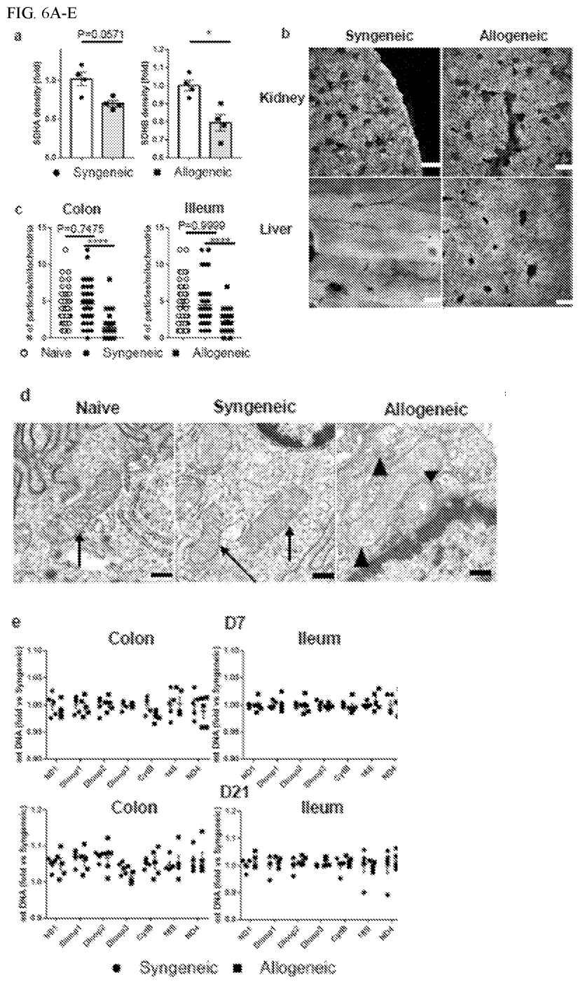

Complex II of mitochondria contains other SDH subunits in addition to SDHA. We therefore next examined SDHA protein levels after HCT to determine whether the SDH dysfunction was due to reduced SDHA protein levels. Mitochondrial proteins were isolated from colonic IECs post HCT and levels of ETC-related proteins (complex I to V) were determined as controls. Reduced SDHA protein levels were observed in IECs from allogeneic animals at 7 d and 21 d post HCT, but no differences were observed in the levels of the other mitochondrial complexes (FIGS. 3a and FIG. 6a).

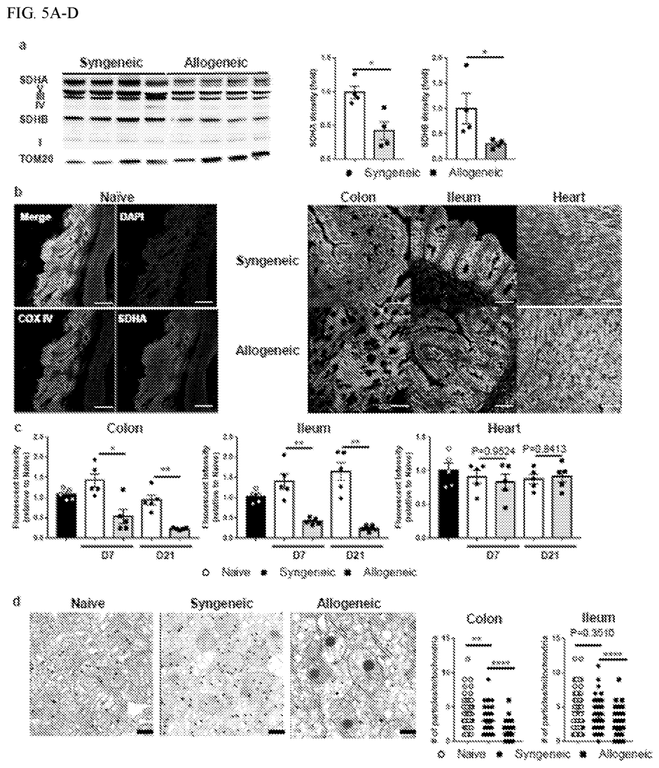

To further validate reduction in SDHA levels, we performed immunofluorescent staining of GI-GVHD lesions in the colon and ileum post HCT and analyzed the fluorescent intensity in an operator blinded manner. The SDHA expression levels were significantly lower in the intestines from allogeneic animals (FIGS. 5b and c). By contrast, the non-GVHD target organs, heart, liver, and kidney demonstrated no differences in the expression of SDHA between syngeneic and allogeneic recipients (FIGS. 5b and FIG. 6b). Finally, to definitively demonstrate that the reduction in SDHA is not a consequence of reduction in mitochondria, we stained SDHA with gold particles post allo-HCT and visualized the gold particles using transmission electron microscopy (TEM). The quantification of absolute gold particles in TEM in a blinded manner. Mitochondria in allogeneic IECs had significantly fewer SDHA gold particles when compared with IECs from naïve or syngeneic animals harvested on days 7 and 21 post HCT (FIG. 5d and FIG. 6c). The noted defects with OXPHOS and mitochondrial respiration in the IECs of allogeneic animals were however associated with significant blunting and loss of discernable cristae in the mitochondria compared to those in the IECs from syngeneic animals (FIG. 6d)¹⁷. Nonetheless, the numbers of mitochondria as measured by mitochondrial DNA copy numbers were similar in IECs from syngeneic and allogeneic animals post HCT (FIG. 6e). Collectively, these results demonstrate that mitochondrial complex II component, SDHA, levels were reduced in allogeneic IECs and was associated with changes in the shape of the cristae but not in the quantity of the mitochondria.

### Example V. Reduction of SDHA in IECs is Specific for T Cell Mediated Intestinal Immunopathology

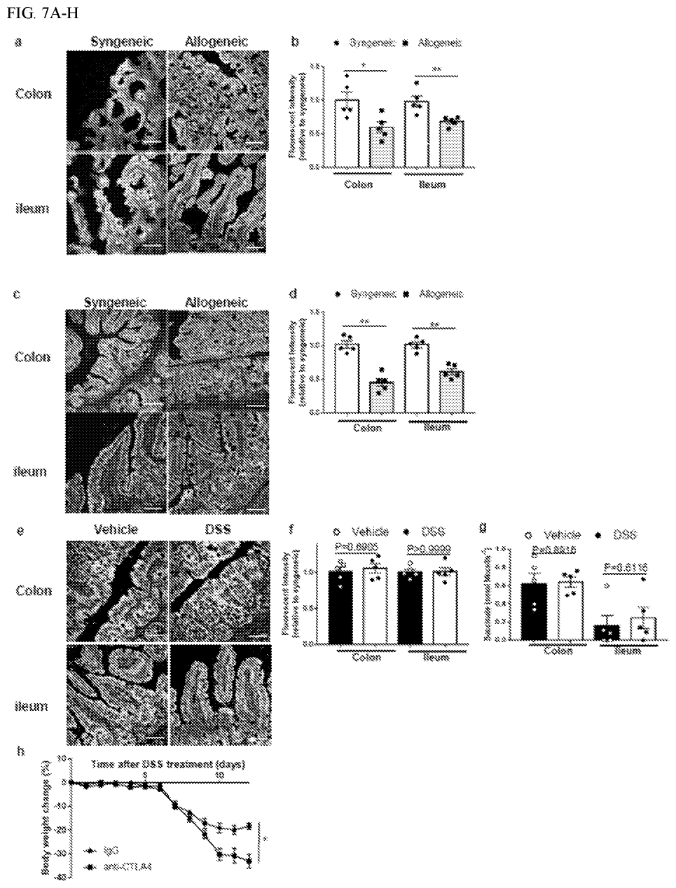

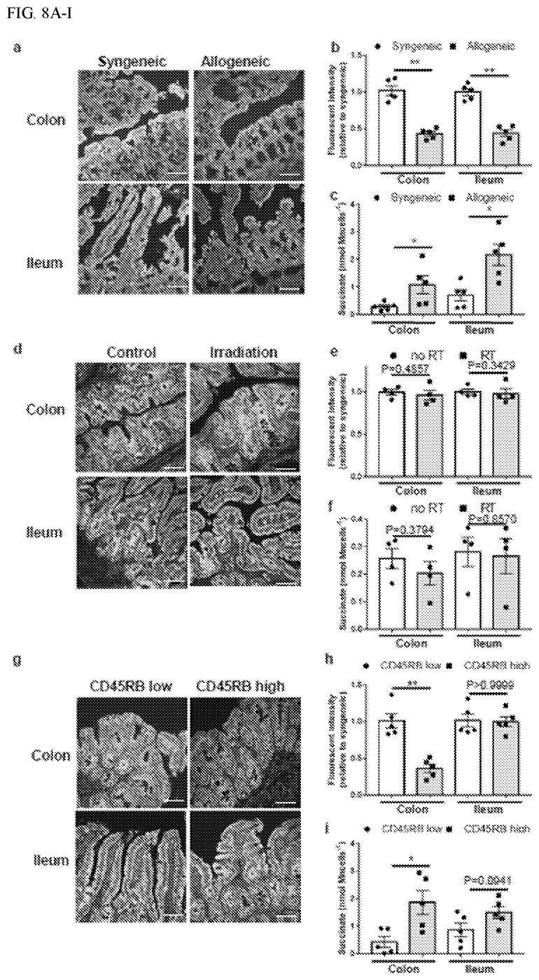

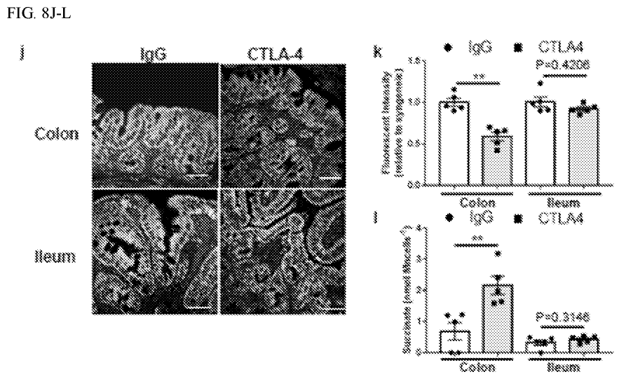

Next, we first explored whether the reduction in SDHA is germane to any type of intestinal damage, such as conditioning with irradiation or is limited to T cell mediated GVHD in a strain independent manner. To first rule of strain and model dependence in the induction of intestinal GVHD, we next utilized the lethally irradiated major histocompatibility complex (MHC)-matched, multiple host minor histocompatibility antigens (mHAgs)-mismatched C3H.SW→C57BL/6 allogeneic HSCT model of GVHD as in Methods. The IECs harvested from the intestines of the allogeneic recipients showed significantly lower SDHA levels (FIGS. 7a and b). To determine whether irradiation dependent conditioning was critical, we next utilized well-established chemotherapy-based conditioning regimen consisting of treatment with busulfan and cyclophosphamide. The IECs harvested from allogeneic animals, once again demonstrated significantly lower levels of SDHA (FIGS. 7c and d). We next analyzed whether allorective T cells alone were sufficient to cause reduction in SDHA independent of conditioning. To this end we utilized the fourth model system, the MHC-haploidentical, non-irradiated parent into F1, the B6→B6D2F1, where alloreactive donor T cells cause GVHD despite absence of any conditioning. Allogeneic IECs once again demonstrated significantly reduced levels of SDHA (FIGS. 8a and b). Furthermore, the reduction in SDHA was associated with the observed metabolic abnormality, the accumulation of succinate in the IECs of allogeneic mice (FIG. 8c).

It is possible that any non-T cell mediated damage of the host IECs could also lead to reduction in their SDHA. Therefore, we irradiated mice with escalating doses to cause intestinal damage but without HCT to induce radiation induced colitis. The IECs harvested 7 days after irradiation did not demonstrate a reduction in SDHA levels nor an increase in succinate levels when compared to non-irradiated controls (FIG. 8d-f) suggesting that unlike alloreactive T cell mediated GVHD, radiation induced colitis did not alter SDHA in the IECs.

We next determined whether the metabolic impact of the reduction of mitochondrial complex II component SDHA in the IECs with concomitant increase in succinate was also germane to non-alloreactive T cell mediated immunopathology, such as in autoimmune T cell mediated colitis. We utilized the well-characterized autoimmune T cell mediated colitis model wherein colitis is caused by transferring WT B6 CD45RB^{high} naïve T-cells into B6 recombination activating gene 1 deficient (RAG-1^{-/-}) hosts. Twelve weeks after T-cell transfer, IECs were harvested from CD45RB^{high} T-cell recipients and control CD45RB^{low} T-cell recipients. Colonic IECs from the CD45RB^{high} group showed significantly lower levels of SDHA and higher levels of succinate accumulation than the colonic IECs from the control CD45RB^{low} T-cell recipients (FIG. 8g-i).

To further analyze the specificity to T cell mediated damage, we next induced colitis by chemical damage. To this end we next utilized the well-established dextran sulfate sodium (DSS)-chemical induced inflammatory colitis model system. In contrast to autoimmune T cell mediated colitis, twelve days after DSS treatment, the colonic IECs from DSS colitis-induced animals demonstrated similar SDHA and succinate levels as the control animals (FIG. 7e-g). These data demonstrate that the specific IEC metabolic abnormality, the reduction in SDHA with accumulation of succinate, was observed exclusively in T cell mediated autoimmune colitis or alloimmune GVHD but not in radiation or chemical colitis.

We further explored whether these observations were broadly applicable to other clinically relevant T cell mediated colitis diseases. T cell dependent immune checkpoint blockers (ICB) have emerged as major form of therapy against many cancers. Amongst the adverse effects of this therapy, the unleashing of T cell mediated colitis is a major toxicity from these therapies, especially with the use of anti-CTLA-4 therapy¹⁸. Therefore, we next explored whether IECs from ICB mediated T cell colitis demonstrate the aforementioned metabolic abnormality, i.e. reduction in SDHA with an increase in levels of succinate. To this end we utilized the recently developed experimental model for ICB mediated colitis^{19,20} and administered anti-CTLA-4 antibody before treatment with low dose DSS to induce ICB mediated colitis^{19,20}. The anti-CTLA-4 treatment induced severe colitis with significant body weight loss, and the colonic IECs demonstrated reduced SDHA with increase in succinate than the control IgG treated animals (FIG. 8j-l and FIG. 7h). Collectively, these findings from eight distinct in vivo models demonstrate that T cell mediated intestinal immunopathology cause a reduction in mitochondrial complex II component SDHA with a resultant increase in succinate in their target cells, the IECs.

### Example VI. Functional Relevance of Reduction in SDHA in IECs

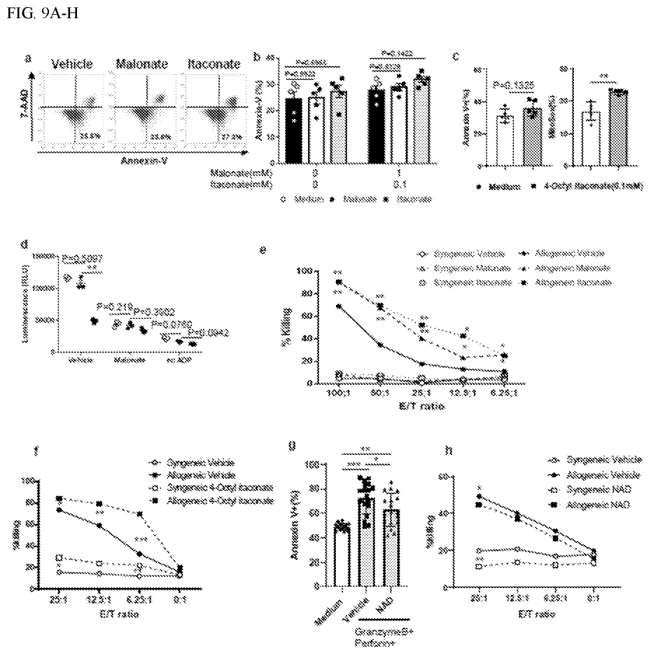

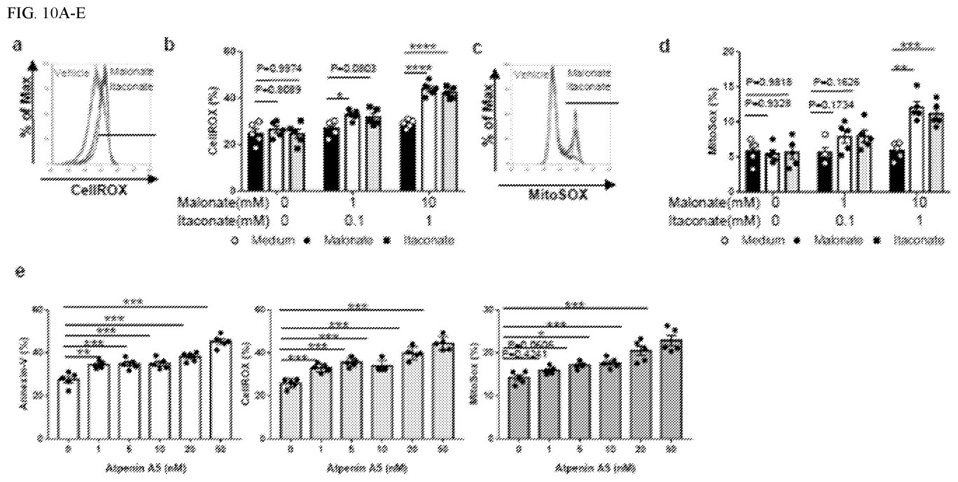

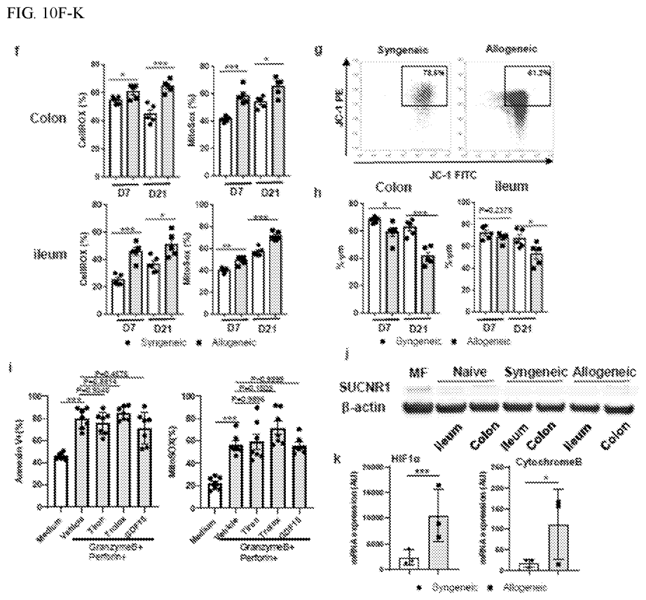

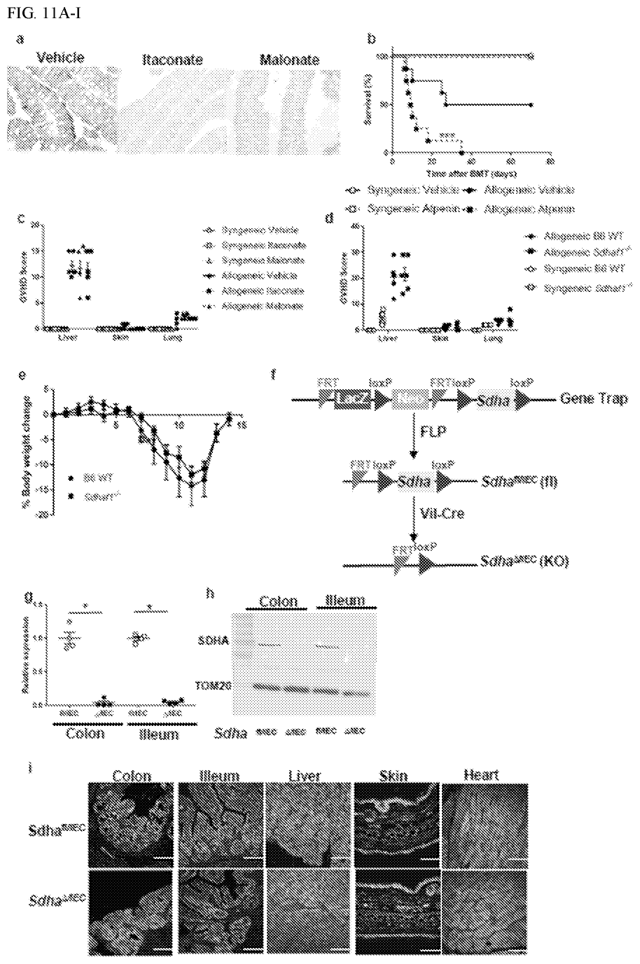

We next explored the functional relevance of SDHA in the IECs when they are targeted by T cells. We treated primary colonic epithelial cells (PCECs) from B6 WT animals with well-characterized SDH inhibitors, namely a cell-permeable ethyl ester derivative of itaconate, dimethyl malonate and 4-octyl itaconate that has less off-targets effect than ethyl ester derivative of itaconate21-. We utilized concentrations of these agents that were non-toxic and yet inhibited SDHA (FIG. 9a-c). SDHA inhibition promoted production of reactive oxygen species (ROS) in mitochondria (mitoSOX; FIG. 5c) and in the cytoplasm (CellROX; FIG. 10a-d) at non-cytotoxic doses. Further to rule out non-specific effects of itaconate and malonate, we also utilized atpenin A5, another known inhibitor of mitochondrial complex II (acts at the interface between SDHB, C, and D)²². Similar to treatment with malonate, itaconate and 4-octyl itaconate, treatment of PCECs with atpenin A5 also ROS production (FIG. 10e). These data suggested that reduction in complex II functions in PCECs increases ROS and might predispose to apoptosis. To assess this possibility, we next determined whether the reduction of SDHA in allogeneic IECs was similarly associated with an increase in mitochondrial ROS. IECs harvested from the colon and ileum of allogeneic mice showed higher mitochondrial ROS levels than IECs from the syngeneic mice (FIG. 10f). We treated isolated mitochondria with malonate to measure ATP production from a non-SDH-related pathway. Mitochondria from syngeneic and naïve animals showed similar levels of ATP production, whereas mitochondria from allogeneic animals produced less ATP, but this difference diminished after malonate treatment (FIG. 9d). Furthermore, IECs from allogeneic mice demonstrated significantly lower mitochondrial membrane potentials ψm than IECs from syngeneic IECs mice (FIGS. 10g and h), consistent with the defect in mitochondrial respiration and OXPHOS.

To functionally demonstrate that inhibition of SDHA leading to ROS generation in the IECs enhanced their sensitivity to T cell mediated apoptosis, B6 PCECs were pretreated with malonate or at non-apoptosis inducing concentrations and then incubated as targets in a chromium release cytotoxic killing assay with primed T cells. PCECs pre-treated with malonate or itaconate showed significantly higher levels of cell death compared with diluent treated PCECs when co-cultured with allo-primed T cells. In contrast the PCECs that were treated with malonate or itaconate or diluent control and cultured with syngeneic T cells (FIG. 9e) showed no increase in apoptosis. The 4-octyl itaconate showed the similar to itaconate, enhance susceptibility to T cell mediated cytotoxicity (FIG. 9f). These data demonstrate that reduction or inhibition of SDHA generates mitochondrial ROS in IECs and enhanced their susceptibility T cell mediated cytotoxicity.

Next we determined the effect of growth and differentiation factor15 (GDF-15) that promotes the tissue tolerance, and ROS scavengers such as Tiron and Trolox^{23,24} regulating the sensitivity of IECs to T cell mediated apoptosis. PCECs were pre-treated with GDF15, Tiron and Trolox and incubated with GranzymeB and peforin to rule direct effects of these agents on T cells as a confounding variable. The pre-treatment with GDF15, Tiron and Trolox ROS did not reduce apoptosis of PCECs induced by GranzymeB and perforin (FIG. 10i). Next, because the NAD/NADH ratio changed in IECs from allogeneic animals (FIG. 1e), we investigated whether nicotinamide adenine dinucleotide (NAD) supplementation regulated T cell mediated apoptosis of IECs. PCECs pre-treated with NAD were incubated with GranzymeB and perforin, or used as targets in vitro allogeneic CTL assay. Pretreatment of PCECs with NAD reduced apoptosis caused by Granzyme B and perforin (FIG. 9g) and by the allogeneic T cells in vitro allogeneic cytotoxic assay (FIG. 9h). Succinate is known to be pro-inflammatory²⁵. Therefore, we next explored that whether the accumulation of succinate in IECs had any paracrine effects on them. To the end, we determined whether IECs express the succinate receptor (SUCNR1) that is utilized for macrophages to sense succinate and turn them into the inflammatory macrophages²⁶. We harvested IECs from naïve, syngeneic recipient mice and allogeneic recipient mice and analyzed for expression of SUCNR1. In contrast to macrophages, IECs did not express SUCNR1(FIG. 10j). These data suggest potential lack of paracrine effect by the succinate released from the IECs. We next assessed whether the accumulation of succinate might have any potential autocrine effects. To this end, we explored for expression of hypoxia inducible factor-1α(HIF1α) and its targeted genes that are known to be induced by succinate²⁵. Consistent with the effects on macrophages, IECs from allogeneic mice showed increased expression of HIF-1α and its target gene such as cytochrome B (FIG. 10k).

### Example VII. SDHA in IECs Regulates Severity of T Cell Mediated Intestinal Immunopathology

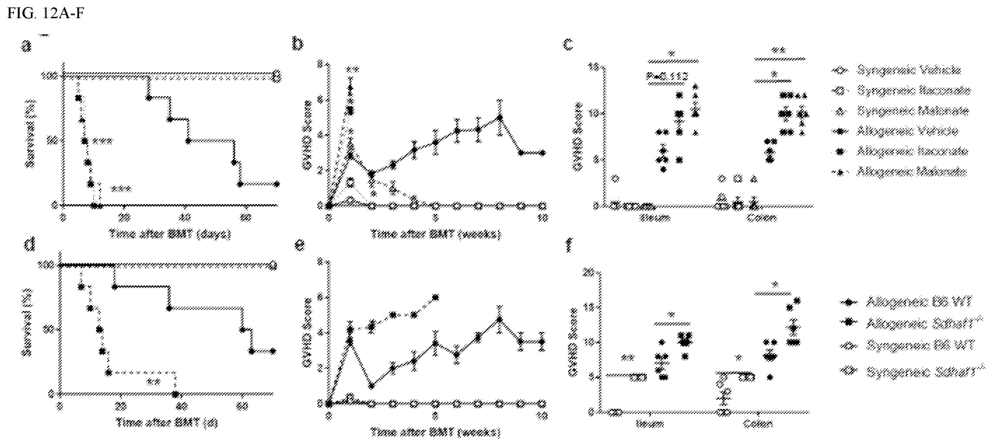

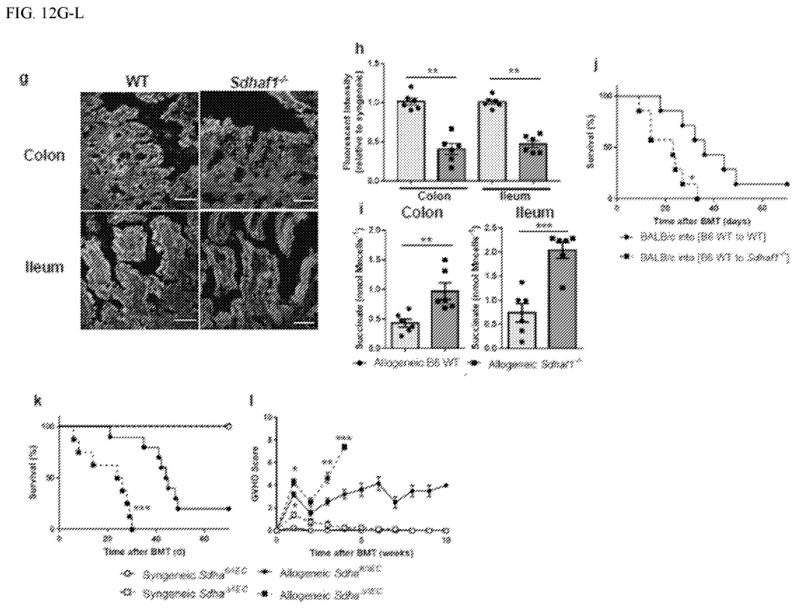

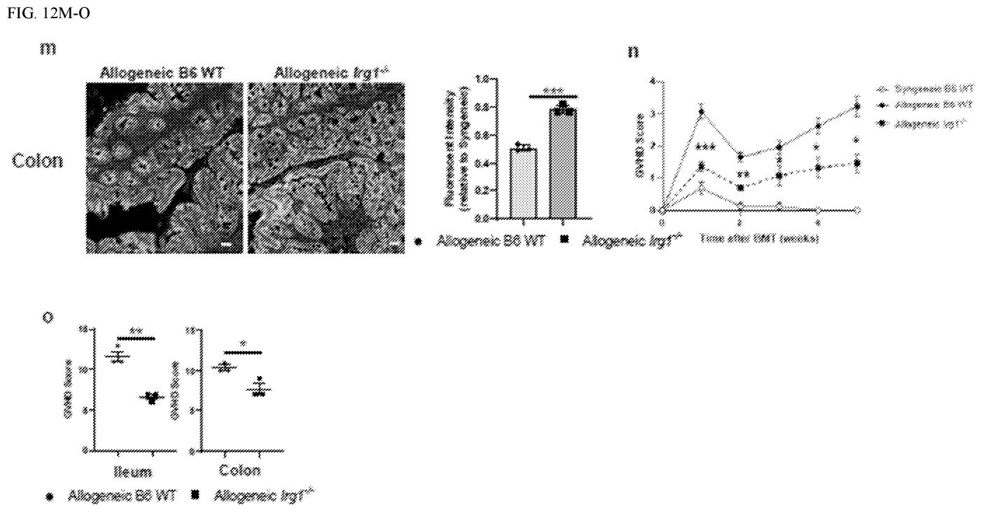

Because in vitro data suggested that reduction of SDHA in enterocytes reduced their ability to withstand T cell mediated cytotoxicity, we next examined the role of SDHA in the enterocytes in vivo, in regulating the severity of alloreactive T cell mediated intestinal GVHD. To this end, we took multiple complementary, but distinct approaches. First, we utilized three distinct chemical inhibitors of SDHA, namely malonate, itaconate and atpenin A5. They were administered orally by gavage to naive B6 WT and the IECs from colon and ileum were harvested 12 h after gavage and were stained for SDH enzymatic reactions. The doses at which they inhibited SDHA function 12 h after in vivo administration (FIG. 11d) were then utilized for analyzing the effect of SDHA inhibition on GI GVHD. Briefly, the recipient animals were gavaged either diluent control or itaconate or malonate or atpenin A5 every other day starting on day 0 post HCT. All of the syngeneic mice survived regardless of treatment, but the allogeneic mice receiving itaconate or malonate or atpenin A5 died significantly faster, and had more severe GVHD symptoms than vehicle treated allogeneic mice (FIG. 12a-b and FIG. 11b). Histopathological analysis confirmed increased GVHD severity in the colon and ileum, but not in the liver, skin, and lungs, of the allogeneic recipients (FIG. 12c and FIG. 11c).

Because chemical inhibitors could have off-target effects, to further confirm the in vivo role of SDHA in GVHD, we next resorted to genetic deficiency models. We used SDH assembly factor 1 deficient B6 mice (Sdhaf1^{-/-}) as recipient in allo-HSCT. These animals have unstable SDH with 20-50% the SDH activity compared with WT B6^{27 28}. Littermate WT and Sdhaf1^{-/-}recipients were utilized to rule out potentially confounding effects from endogenous microbiome changes. Both WT and Sdhaf1^{-/-}syngeneic animals showed no mortality and or GVHD demonstrating lack of non-T cell mediated toxicity. By contrast allogeneic Sdhaf1^{-/-}B6 mice showed greater severity of GVHD and significantly greater mortality when compared with allogeneic WT B6 littermate controls (FIGS. 12d and e). Histopathological analysis of the GI tract confirmed greater GVHD severity in the allogeneic Sdhaf1^{-/-}mice than allogeneic WT littermates (FIG. 12f and Supplementary FIG. 6d). Furthermore, the intestines from Sdhaf1^{-/-}recipients showed lower SDHA levels than those from WT littermates 7 d post allo-HCT (FIGS. 12g and h). Succinate accumulation greater in IECs harvested from allogeneic Sdhaf1^{-/-}recipients reflecting insufficient SDH activity (FIG. 6i).

It is possible that reduction of SDHA in IECs might make them generally more susceptible to any intestinal damage because of reduced fitness and might not be unique only to T cell mediated pathology. To rule out this possibility we induced chemical colitis with DSS in Sdhaf1^{-/-}B6 animals and compared severity with B6 WT littermates. The Sdhaf1^{-/-}and the WT animals demonstrated similar changes in body weight and colitis severity demonstrating that the reduction in SDHA predisposed the enterocytes only to T cell mediated cytotoxicity but did not make them more susceptible to non-T cell (i.e. DSS) mediated damage (FIG. 11e).

The above approaches, given the systemic effects, do not distinguish potential confounding effects of SDHA activity in host hematopoietic, immune and other cell types from the direct effects on intestinal epithelial target cells. Therefore, first to determine whether SDHA expression exclusively in the host target non-hematopoietic cells is critical for regulation of GI GVHD, we generated BM chimeric mice wherein low SDH activity was restricted to the non-hematopoietic recipient cells. The [WT B6Ly5.2→WT B6] and [WT B6Ly5.2→Sdhaf1−/−B6] chimeric animals were generated and utilized three months later as recipients of allogeneic HCT. The allogeneic [WT B6Ly5.2→Sdhaf1−/−B6] chimeras demonstrated significantly shorter survival when compared with WT B6Ly5.2→WT B6 recipients (FIG. 12j).

Next, to eliminate germline effects of Sdhaf1^{-/-}and to demonstrate that the enhanced intestinal pathology is caused only by deficiency of SDHA in the enterocytes, and not confounded by effects from other host non-hematopoietic cells, we generated IEC specific SDHA deficient B6 animals. To this end we obtained Sdha gene trap, floxed it, and then these mice containing loxP-flanked Sdha were crossed with Villin-Cre mice to generate mice specifically lacking Sdha in IECs (Sdha^{Δ/IEC}; FIG. 11f). Efficient reduction of SDHA exclusively in the IECs from the colon and ileum was confirmed by both immunoblot and quantitative reverse transcription PCR (RT-qPCR; FIGS. 11g and h) in Sdha^{Δ/IEC} mice when compared to the Sdha^{fl/IEC} animals. Importantly, both the Sdha^{Δ/IEC} and Sdha^{fl/IEC} mice expressed similar levels of SDHA protein in the heart, liver, and skin, and thus validated enterocyte specific reduction of SDHA without differences in other tissues (FIG. 11i). The WT Sdha^{fl/IEC} and IEC specific KO Sdha^{Δ/IEC} littermate animals were utilized as recipients and transplanted from with BM and T cells from syngeneic B6 or allogeneic BALB/c donors as in Methods. Allogeneic IEC specific KO Sdha^{Δ/IEC} recipients demonstrated significantly greater mortality and clinical severity of GVHD when compared to the allogeneic WT Sdha^{fl/IEC} littermate animals (FIGS. 12k and l).

Next, we analyzed whether conditions where endogenous SDHA levels can be increased would reduce T cell mediated intestinal damage. To this end we utilized immunoresponsive gene 1 deficient B6 mice (Irg1^{-/-}) as recipients. Irg1 enhances endogenous itaconate production (inhibitor of SDHA)²⁹, thus resulting in increased levels of SDHA. Therefore, we hypothesized that the absence of Irg1 would result in improved levels of SDHA after allo-BMT and result in reduction in intestinal damage from GVHD. Consistent to the hypothesis, allogeneic Irg1^{-/-}mice demonstrated greater SDHA level, reduced clinical and intestine severity from GVHD when compared to littermate WT recipients (FIG. 12m-o). These results indicate that SDHA expression exclusively in IECs regulates severity of T cell mediated intestinal GVHD pathology.

### Example VIII. In Vitro and In Vivo Mechanism of T Cell Mediated Reduction of SDHA in Enterocytes

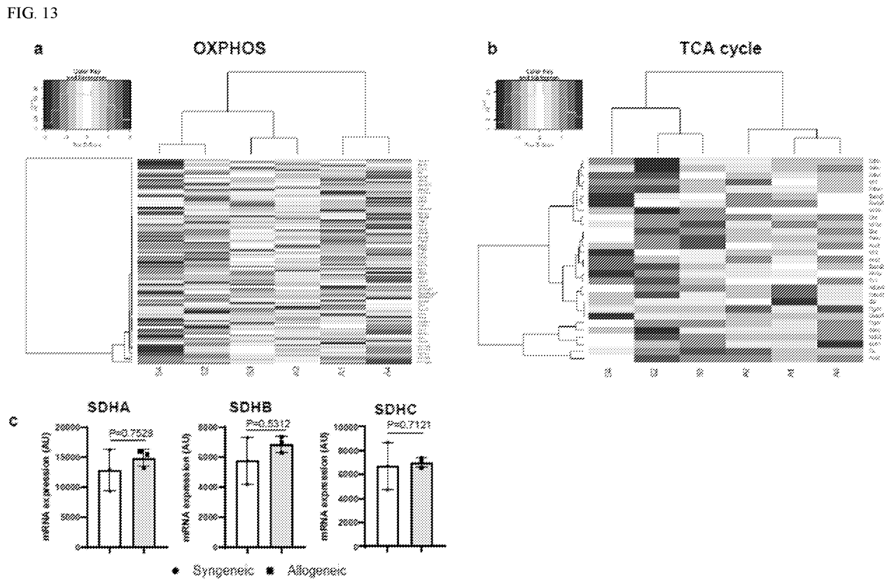

To determine mechanism of reduction in SDHA, we performed transcriptomics of IECs harvested from syngeneic and allogeneic recipients 7 days after HCT and analyzed for the expression of OXPHOS and TCA cycle genes (see Methods). We found no significant differences between syngeneic mice and allogeneic mice in OXPHOS and TCA cycle genes (FIG. 13a, b) expression. Furthermore, there was no significant difference in expression of mitochondrial complex II subunits, including SDHA, SDHB and SDHC between the groups (FIG. 13c). These data suggested that the reduction in SDHA protein levels in the IECs from allo-recipients may likely be from post-transcriptional processes.

To investigate the mechanism of T cell-dependent reduction in SDHA, we next analyzed whether the reduction of SDHA in IECs was dependent on direct contact with T cells. The PCECs targets were incubated with T cells primed with syngeneic or allogeneic stimulators in a transwell assay. PCECs cultured with primed syngeneic T cells, as expected, did not show any increase in apoptosis regardless of contact. By contrast, the PCECs that were in direct contact with allo-primed T cells, showed greater apoptosis when compared to those separated by transwell membrane from the allo-primed T cells (FIG. 13a). Importantly when the PCEC targets were isolated and stained with SDHA from these transwell experiments after 12 hours of culture, only the PCECs that were in direct contact with allo-primed T cells showed reduction in SDHA when compared to those separated by transwell from allo-primed T cells or syn-primed T cells (FIG. 13b). These data demonstrated that reduction of SDHA in the IEC targets required direct contact by T cells primed against allo-antigen and that soluble mediators such as inflammatory cytokines were not the cause for reduction in SDHA levels in the IEC targets.

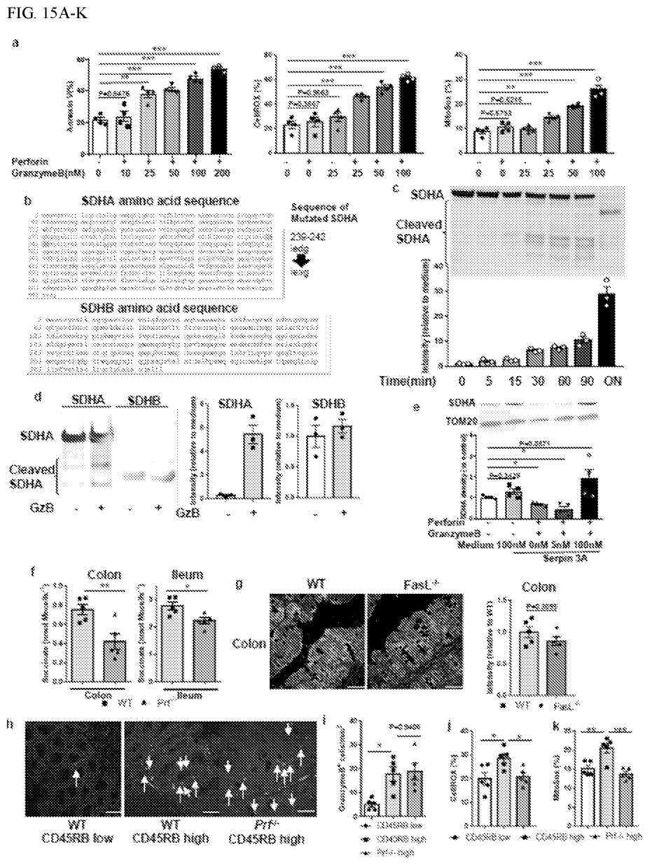

We therefore reasoned that T cell cytotoxic pathways might be the cause for reduction in SDHA. Previous reports have suggested that granzyme B (GzmB) degrades mitochondrial complex proteins in parasites²³. We therefore explored whether GzmB was required for reduction in SDHA. We cultured PCECs with increasing concentrations of recombinant perforin (PFN) and/or GzmB. The combination of both GzmB and PFN caused a decrease in SDHA levels in the PCECs when compared with control, PFN, or GzmB alone (FIG. 7c, FIG. 15a). Furthermore, mitochondrial ROS production was increased when treated with both GzmB and PFN (FIG. 15a). These data suggest that the requirement of perforin dependent intracellular transport of GzmB is required for reducing SDHA.

To further dissect the direct mechanisms of GzmB mediated reduction of SDHA, we next hypothesized that GzmB directly cleaves SDHA once it gains access to the IEC cytoplasm following effects of perforin. SDHA, but not SDHB, demonstrates the putative GzmB recognition Ile-Glu-Asp↓Gly site (FIG. 15b)³⁰. Mitochondria isolated from IECs were treated ex vivo with PFN and GzmB. Treatment with GzmB caused proteolysis of SDHA with a reduction in SDHA with an increase in SDHA cleaved fragments (FIG. 15c). Next, to rule out confounding effects from other cellular proteins, and to directly assess proteolysis of SDHA by GzmB, we utilized cell free experimental system. Recombinant SDHA was treated with GzmB and assessed for its full length and cleaved peptides. Recombinant SDHB was used as control. GzmB caused direct proteolysis of recombinant SDHA but not SDHB (FIG. 15d). Next, to further confirm proteolysis, we mutated SDHA protein at the putative GzmB mediated cleavage site (Ile-Glu-Asp-Gly to Ile-Glu-Ala-Gly, FIG. 15b). As shown in FIG. 7d, the mutation of the cleavage site prevented proteolysis of SDHA by GzmB. To further confirm the direct proteolysis of SDHA we inhibited the activity of GzmB with Serpin 3A, an inhibitor of GzmB³¹. The presence of Serpin 3A prevented the proteolysis of SDHA by GzmB (FIG. 15e). These data collectively demonstrate that T cell-derived GzmB cleaves SDHA and thus reduces its levels in the IECs.

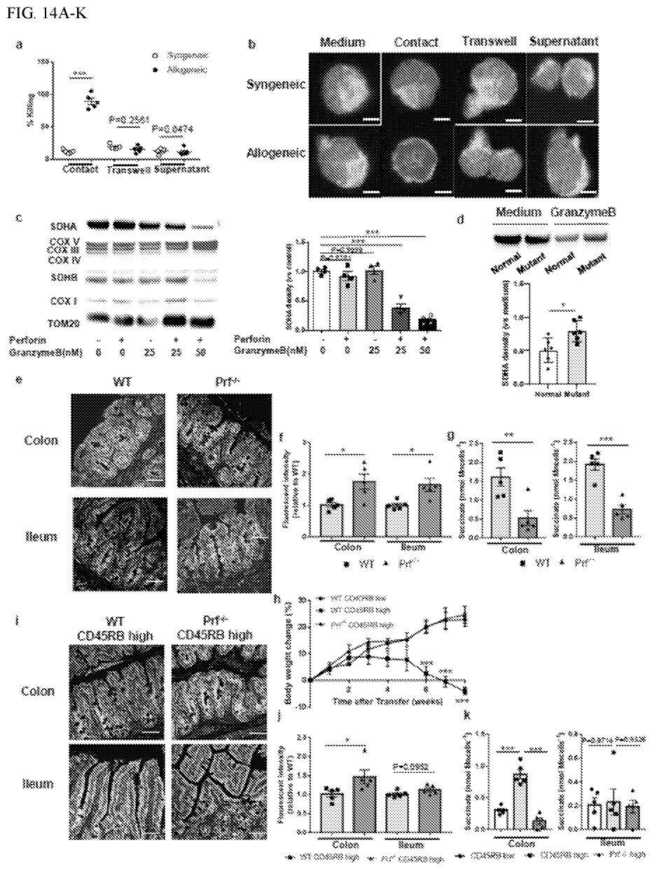

We next evaluated whether the PFN-GzmB dependent cytotoxic pathways is the critical mechanism for in vivo metabolic defect, reduction of SDHA and accumulation of succinate, in the IECs during GVHD and in autoimmune IBD. We transplanted WT or Prf^{-/-}donor T cells from B6 mice along with TCD WT BM cells into allogeneic BALB/c mice and harvested IECs from the transplant recipients on days +7 and +21. The syngeneic recipients once again showed no difference in SDHA levels, while the allogeneic recipients of WT T cells, demonstrated, as above, a significant reduction in SDHA levels with an increase in levels of succinate. By contrast, recipients of allogeneic Prt^{-/-}T cells showed no reduction in SDHA, the levels were similar as in syngeneic animals and also did not demonstrate accumulation of succinate in the IECs harvested on both days 7 and 21 after HCT (FIG. 14e-g and FIG. 15f).

T cells utilize other cytotoxic pathways, including FasL, which could induce target cell apoptosis and cause GVHD. Therefore, to determine whether Fas-Fasl pathway could mediate similar enterocyte damage, we utilized B6 FasL deficient donor T cells with WT BM in the allo-HCT model as above. FasL deficient donor T cell caused similar reduction as WT T cells in allogeneic recipients (FIG. 15g), demonstrating a specific role for T cell PFN-GzmB pathways in reduction of SDHA in the IECs.

To further confirm the in vivo relevance of perforin-granzyme pathway in regulation of intestinal SDHA and disease severity of T cell mediated colitis, we next determined the relevance of PFN-GzmB pathways in mediating the aforementioned metabolic abnormality in autoimmune T cell mediated IBD. The WT or Prf^{-/-}CD45RB^{high} naïve T cells were adoptively transferred into syngeneic RAG1^{-/-}mice to induce colitis. When compared to recipients of CD45RB^{high} WT T cells, recipients of CD45RB^{high} Prf^{-/-}T cells showed a higher body weight, with and no effect on SDHA levels or the levels of succinate in the IECs (FIG. 7h-k). It is, however, formally possible that perforin deficient T cells may demonstrate migratory defects that may reduce their infiltration into colon. To address this, adoptively transferred CD45RB^{high} T cells were tracked and quantified by immunofluorescence, in recipients of both WT and Prf^{-/-}T cells. Both CD45RB^{high} WT and CD45RB^{high} Prf^{-/-}T cells demonstrated similar Gzmb⁺T cell infiltration (FIGS. 15h and i), but a reduction in induction of ROS (FIGS. 15j and k) in the IECs harvested from the recipient of the CD45RB^{high} Prt^{-/-}T cells, consistent with differential effects on SDHA levels and succinate accumulation as noted above. Taken together, these data reveal that T cells directly induce IEC damage and that GzmB/PFN play critical roles in abrogating SDHA levels in vitro and in vivo.

### Example IX. Clinical Correlation of Loss of SDHA in Humans

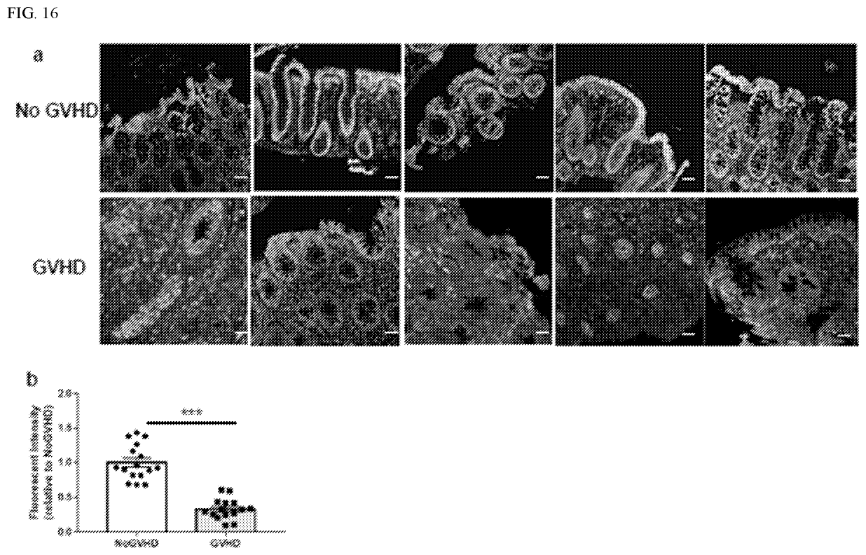

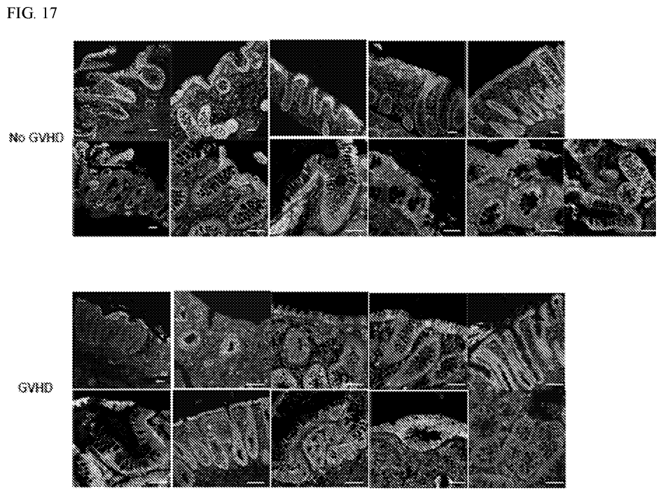

We next determined whether the loss of SDHA has any human, clinical relevance. To this end we obtained intestinal biopsy samples from 31 patients that underwent allogeneic HCT and received similar immunoprophylaxis and were clinically suspected for having lower GI GVHD. They all then underwent GI biopsy for diagnostic confirmation. None of these patients were on mycophenolate mofetil at the time of biopsy. In about half of these patients, biopsy confirmed the clinical diagnosis and in the other half, the biopsy was read as negative for GI GVHD. These 31 samples were stained and quantified for SDHA expression and analyzed in a manner blinded to the clinical and histopathological diagnosis. SDHA expression was dramatically reduced in the colonic biopsy samples from patients that were histopathologically confirmed as GI GVHD when compared with those who were pathologically considered not to have GI GVHD (FIG. 16a, FIG. 17). The SDHA fluorescence intensity levels was statistically significant between GVHD and non-GVHD samples (FIG. 16b).

### Example X

This example provides a discussion of Examples I-IX.

The metabolism of immune cells, such as T cells is dysregulated in immune diseases. T cells play a key role in alloimmune, autoimmne and iatrogenic colitis. The critical T cell and other immune cell dependent mechanisms between these various T cell mediated diseases such as GI GVHD, IBD and ICB are being increasingly recognized and understood. However, the tissue intrinsic mechanisms the immune targets, the IECs, remain uncharacterized. Importantly, whether the target cell intrinsic metabolic aberrations can regulate the overall disease severity of colitis remain unknown. In the experiments described herein, we demonstrated a specific metabolic aberration in mitochondrial complex II of the IECs that is common to and exclusive for many T cell mediated diseases of the intestinal tract. We showed that the IECs accumulate succinate as a consequence of reduction in their mitochondrial complex II component, SDHA. This reduction in SDHA caused an enhanced sensitivity of the IECs to T cell mediated cytotoxicity, thus serving as an IEC intrinsic metabolic checkpoint that regulated disease severity independent of direct effects of immune cells. The inhibition of SDHA. IECs altered their bioenergetics, decreased OXPHOS without a compensatory increase in glycolysis, which led to reduced O2 utilization, ATP generation and an enhanced ROS production. GzmB/PFN from the reactive T-cells worked together to lyse SDHA in IECs. PFN disrupt host cells to deliver the GzmB, which degrades SDHA and disrupts mitochondrial complex II and thus inhibits both TCA cycle and mitochondrial respiration of the IECs. Utilizing combination of metabolic, genetic, chemical loss and gain of function approaches we demonstrated that SDHA reduction in IECs is the critical target tissue intrinsic common pathway for T-cell mediated colitis, specifically in five models of GI GVHD, two (DSS and CD45RB^{high}) models of autoimmune colitis, and a CTLA-4Ig ICB mediated colitis models. We further validated the observation in human samples of GI GVHD. These data identify tissue intrinsic SDHA as novel target to mitigate multiple T cell mediated intestinal immunopathologies³². Nonetheless, the specificity of SDHA to granzyme B in vivo will need to be assessed directly with mutated, non-cleavable SDHA expression in these in vivo model systems.

Succinate is a metabolic intermediate that is converted into fumarate by SDHA in the TCA cycle. Succinate plays an important role as an inflammatory signal, or as a source for reverse electron transport (RET) in the context of preserved SDH functions³³. In our study the impact on complex II from reduction of SDHA demonstrates that both TCA cycle and mitochondrial respiration are affected³⁴. Eukaryotes mainly produce energy as ATP via glycolysis in the cytoplasm or TCA cycle and ETC in mitochondria depending on cell status and oxygen levels. T cells (other immune cells as well), when activated show an increase in both glycolysis and OXPHOS^{35 36}. By contrast, the intestinal immune targets, IECs show a loss of their major energy pathway, TCA cycle, without a compensatory change in glycolysis leading to a metabolic shutdown, which likely contributes to their greater susceptibility to apoptosis. At homeostasis, IECs efficiently utilize β-oxidation via SCFAs, especially butyrate, and SDHA serves as a part of TCA cycle enzyme and as a part of ETC¹⁵. However, in the context of T cell colitis, not only did glycolysis levels show no changes, but isotope tracing assay demonstrated that neither glucose, nor odd chain carnitine nor glutamine supplementation failed to complement or compensate the energy production in IECs. These results show that IECs critically depend on OXPHOS and that neither enhanced glycolysis nor anaplerosis mitigated the defects from the reduction of SDHA. Energy insufficiency in IECs enhanced susceptible to T-cell mediated cytotoxicity was observed in vivo with both chemical inhibition and IEC specific genetic ablation of SDHA studies. Thus succinate accumulation in IECs caused by SDHA reduction, i.e defect in mitochondrial complex II, is distinct from the mitochondrial defects observed in other contexts such as ischemia reperfusion model, LPS activated macrophages or in other mitochondrial complex deficiencies^{21 22 37}. However, decreased NADH production by insufficient TCA cycle, a substrate for complex I reaction the starting point of ETC, could also secondarily contribute to further slowing down of the mitochondrial respiration and metabolic shutdown in the IECs caused by pathogenic T cells.

Succinate stimulates macrophages by increasing HIF1αexpression dependent on activating IL-1β²⁵. Our data suggests that the accumulated succinate in IECs may act in an autocrine manner and enhance HIF1αdependent pathways, but not in paracrine manner because of absence of detectable level of SUCNR1 on IECs.

Acute GVHD, intriguingly, affects only the GI tract, liver and skin, the mechanisms for which remain unclear. Nonetheless, clinically, GI GVHD is the major cause for fatality from acute GVHD. Our data demonstrate that IECs when targeted by alloimmune cells across multiple models, regardless of type of allo-disparity and type of conditioning regimen, demonstrated reduction in SDHA with a concomitant increase in succinate. Importantly this reduction in SDHA was observed in human IECs only in those who had histopathologically confirmed GI GVHD. These data suggest that SDHA levels in IECs may serve as a marker for GI GVHD. Future, well designed human studies will evaluate its role as a potential diagnostic or prognostic biomarker for GI GVHD. Importantly, the reduction in SDHA was observed on in the IECs but not in the non-target organs of GVHD such as the kidneys demonstrating that specificity of T cell mediated cytotoxicity as a critical feature. This is further corroborated by lack of the effect on SDHA/succinate in the context of conditioning initiated damage such as from radiation induced colitis in the absence of alloreactive T cells. Intriguingly this specific mitochondrial abnormality was also not observed in other known GVHD target organs, namely liver and skin (data not shown), which are also caused by alloreactive T cells. This suggests that the dominant metabolic pathways and their alterations in IECs may be distinct from other GVHD target tissues.

The specificity of mitochondrial complex II component, SDHA, dysregulation was also observed only in the context of T cell mediated autoimmune colitis and not in a chemically induced (DSS) colitis model wherein colitis is induced by both innate and adaptive immune cells³⁸. Additionally, it was observed only when T cells were activated by checkpoint blockade in the context of low dose DSS resulting in colitis. This model reflects ICB mediated colitis, a major complication of the checkpoint blocker therapies against cancer^{19,20}. Importantly, inhibition of SDHA aggravated autoimmune and ICB mediated colitis. Thus the dysregulation of complex II of mitochondria in the IECs is a critical metabolic checkpoint for alloimmune, autoimmune and iatrogenic T cell mediated colitis. The reduction in the ability of the IECs to tolerate T cell mediated attack might be a consequence of a reduction in the general fitness of the cells is the absence of reduced SDHA. However, the lack of increase in disease severity from reduction of SDHA in IECs in non-T cell mediated intestinal pathologies such as radiation or chemical colitis suggests that the lack of tissue tolerance is not a global loss of fitness, but unique to T cell mediated damage.

Previous seminal studies have demonstrated that granzyme B targets mitochondrial complexes and several other substrates in bacteria and other cell types³⁹⁻⁴². Thus perforin-granzyme B mediated cell death of target cells could be the result of net effect on several substrates that are targeted by granzyme B. Nonetheless, in IECs, targeting of SDHA by granzyme B appears to be critical for modulating T cell mediated intestinal toxicity as demonstrated by greater apoptosis and damage in vivo in the absence, or inhibition, of SDHA in GVHD, IBD and ICB colitis. In addition, the increase in the intestinal toxicity was not observed in non-T cell mediated colitis model systems such as with DSS and radiation. Furthermore, this deficiency does not seem to enhance the susceptibility of other GVHD target tissues such as liver or skin or non-target tissues like kidney to T cells (Supplementary FIGS. 2 and 3). It is tempting to speculate that distinct target tissues may be uniquely susceptible to granzyme B depending on their preferred, intrinsic metabolic pathways.

The role of perforin/granzyme in causing disruption of mitochondrial complex II subunits in IECs suggests these pathways may be germane to both CD8⁺and also to CD4⁺mediated colitis⁴³⁻⁴⁵. Furthermore, because NK cells utilize similar cytotoxic pathways, it is tempting to speculate that when appropriately primed, they may cause similar effects on IECs. Much also remains to be understood about the target tissue subtypes. Our studies utilized CD326⁺enterocytes, but the intestinal tract has several subtypes and in distinct phases of differentiation, such as ISCs, Paneth cells, tuft cells, Goblet cells etc. The dominant metabolic pathways, bioenergetics for many of these cell subsets remain to be understood.

These data also raise several interesting possibilities with potentially significant implications. For example, it is possible that T cell mediated autoimmune diseases afflicting other organs may share this common pathway. These data align with the notion that factors that promote tissue tolerance such in infectious diseases and extend the concept to non-infectious T cell mediated colitis²⁴, although the mechanism may differ. Nonetheless, they demonstrate for the first time a tissue autonomous role for regulation of non-infectious immune-pathology supporting the concept that optimal therapy for immune mediated diseases will need approaches that promote immune tolerance and tissue resilience. The data thus suggest that mitochondrial SDHA in IECs may represent a novel target to mitigate the severity of GI GVHD, IBD and ICB colitis as an adjunct to T cell immunoregulatory approaches^{27,46,47,48,49,50}.

Furthermore, the converse, inhibiting SDHA in T cell targets may have therapeutic implications for enhancing T cell dependent immunotherapies. For example, it is possible that inhibiting SDHA in cancers where the tumor cells are predominantly dependent on OXPHOS may enhance their susceptibility to graft versus leukemia or ICB mediated cancer immunotherapy. Future studies will need to systematically explore these possibilities. Another intriguing possibility is that SDHA disruption in IECs led to reduced OXPHOS and ETC and caused decreased utilization of O₂, potentially leading to increase in O₂ levels in the intestinal lumen. This increase in O₂ may provide a potential mechanistic explanation for changes in microbiome associated with GVHD and IBD. The change in O₂ levels might lead to dysbiosis with loss of microbial diversity, decrease in commensals such as obligate anaerobes and shift towards to aerobes and pathobionts in the microbiome of patients with GVHD and IBD^{51 52 53 54 55 56 57 58 59 60 61 62 63}.

In summary we identify a novel IEC intrinsic metabolic checkpoint that regulates their sensitivity to T cell mediated cytotoxicity and identify the component of mitochondrial complex II, SDHA, as a potential novel therapeutic target to regulate alloimmune, autoimmune and iatrogenic T cell mediated colitis.

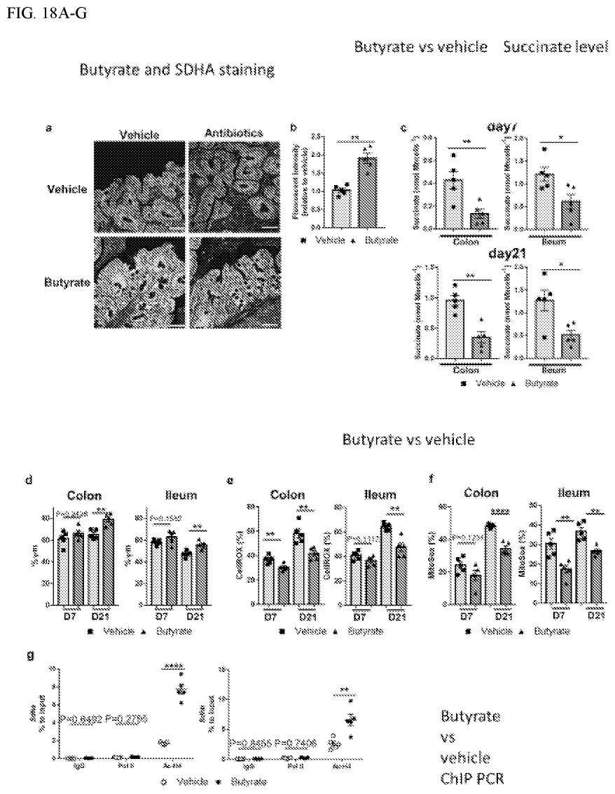

FIG. 18A-D shows the ability of butyrate to reduce IEC related SDHA activity and/or expression; reduce IEC related oxidative phosphorylation; increase IEC related succinate accumulation; and increase IEC related accumulation of perforin dependent granzyme B related to cytotoxic T cell engagement with such IECs.

### Example XI. Materials and Methods

Mice: C57BL/6 (WT B6, H-2K^{b}, CD45.2), B6 Ly5.2 (H-2K^{b}, CD45.1), B6.129S7-Rag1^{tm1Mom}/J(Rag-1^{-/-}), C57BL/6-Prf1^{tm1Sdz}/J(Prf^{-/-}), B6Smn.C3-Faslgld/J (FasL^{-/-}), Sdhaf1^{-/-}, B6. Cg-Tg(ACTFLPe)9205Dym/J, B6.Cg-Tg(Vil-cre) 1000 Gum/J mice, C3H.SW (H-2K^{b}, CD229.1), and C57BL/6N-Acod1em1(IMPC)J/J (Irg1^{-/-}) mice were purchased from the Jackson Laboratory (Bar Harbor, ME, USA). BALB/c (H-2K^{d}) and BDF1 (H-2K^{b/d}) mice were purchased from Charles River Laboratories (Wilmington, MA, USA). C57BL/6N-Sdha^{tm2a(KOMP)Wtsi} mice were obtained from Knock Out Mouse Project (KOMP) repository, University of California, Davis (Davis, CA, USA) and bred to ACTFLPe mice to excise the FRT-flanked region (Supplementary FIG. 6f). Then Sdha^{fl/fl} mice were bred to Vil1-Cre mice to create Vil1-Cre Sdha^{fl/fl} (Sdha^{Δ/IEC}) mice. 8-12 weeks old female mice used for experiments. All mice were kept under specific pathogen-free (SPF) conditions and cared for according to regulations reviewed and approved by the University of Michigan Committee on the Use and Care of Animals, which are based on the University of Michigan Laboratory Animal Medicine guidelines.

Cell culture: C57BL/6 primary colonic epithelial cells (PCEC, C57-6047, Cell Biologics, Chicago, IL) were cultured in compete epithelial growth medium supplemented with insulin-transferrin-selenium, epidermal growth factor, glutamine, antibiotics, antimycotics and fecal bovine serum (#M6621, Cell Biologics). Cells were routinely tested for mycoplasma contamination using MycoAlert (#LT07-318, Lonza, Switzerland). Cells were treated with Mouse active perforin (#APB317Mu01, Cloud-Clone Corp., Katy, TX) and recombinant mouse Granzyme B (#140-03, PeproTech, Rocky Hill, NJ) for 4 to 6 hours in indicated concentrations following manufacturer's recommendations. Cells were incubated with GDF-15 (#8944-GD-025, 500 pg/mL, R&D Systems, Minneapolis, MN), Tiron (#172553, 10 mM, Sigma-Aldrich, St. Louis, MO), Trolox (#648471, 0.5 mM, Sigma-Aldrich), 4-Octyl itaconate (#SML2338, 0.1 mM, Sigma-Aldrich) and NAD (#N8285, 1 mM, Sigma-Aldrich).

Generation of bone marrow (BM) chimeras: WT B6 and Sdhaf1^{-/-}animals were subjected to 1000 cGy total-body irradiation (TBI) from a ¹³⁷Cs source on day −1 and then injected intravenously with 5×10⁶ BM cells from B6 Ly5.2 donor mice on day 0. Donor hematopoietic chimerism was confirmed using a CD45.2 monoclonal antibody 3 months after transplantation.

Hematopoietic cell transplantation (HCT): Splenic T cells from donors were enriched, and the BM was depleted of T cells by autoMACS (Miltenyi Biotec, Bergisch Gladbach, Germany) utilizing CD90.2 microbeads (#130-121-278, Miltenyi Biotec). WT B6, C3H.SW, Sdhaf1^{-/-}, Sdha^{Δ/IEC} , Irg1h−/−B6 and BALB/c animals were used as recipients and received 1,000 cGy (WT B6, Sdhaf1^{-/-}and Sdha^{Δ/IEC}) or 800 cGy (BALB/c) TBI on day −1, respectively, and 1×10⁶ (C3H.SW→C57BL/B6 or C57BL/B6→BALB/c), 3×10⁶ (BALB/c→C57BL/6, Sdhaf1^{-/-}, Sdha^{Δ/IEC}) CD90.2⁺T cells along with 5×10⁶ T-cell-depleted BM (TCD-BM) cells from either syngeneic or allogeneic donors on day 0. [B6Ly5.2→B6] and [B6 Ly5.2→Sdhaf1^{-/-}] animals received 900 cGy TBI on day −1 and were injected intravenously with 3×10⁶ CD90.2⁺T cells and 5×10⁶ TCD-BM from either syngeneic B6 or BALB/c donors on day 0. For the chemotherapy conditioning model, B6 WT mice received busulfan (#B2635, 25 mg kg⁻¹ from day −7 to −4; Sigma-Aldrich) and cyclophosphamide (#C7397, 100 mg kg⁻¹ from day −3 to −2; Sigma-Aldrich) intraperitoneally, and 1×10⁷ (BALB/c→C57BL/6) CD90.2⁺T cells along with 1×10⁷ TCD-BM cells from either syngeneic or allogeneic donors on day 0. For the no-irradiated model, unirradiated B6D2F1 mice were intravenously injected with 10×10⁷ splenocytes from B6D2F1 or B6 donors. Animals received vehicle or dimethyl malonate (DMM, #136441, 0.5 g or 5g kg⁻¹, Sigma-Aldrich, St. Louis, MO, USA), dimethyl itaconate (DI, #09533, 0.25 g or 2.5g kg⁻¹, Sigma-Aldrich) or atpenin A5 (#11898, 0.9 μg or 9 μg kg⁻¹, Cayman Chemical, Ann Arbor, MI, USA) according to manufacturer's instructions by flexible 20-gauge, 1,5-in, intra-gastric gavage needle every other day from day 0. The mice were randomly assigned to syngeneic, allogeneic or treatment groups in each experiment. No mice were excluded from analysis. No statistical methods were used to predetermine sample size. The investigators were not blinded to allocation during experiments and outcome assessment.

Systemic and histopathological analysis of GVHD: Survival after HCT was monitored daily and assessed the degree of clinical GVHD weekly, as described previously⁶⁴. Histopathological analysis of the liver, gastrointestinal (GI) tract, and lung, which are the primary GVHD target organs, was performed as described utilizing a semi-quantitative scoring system implemented in a blinded manner by a single pathologist (C.L.)⁶⁵. After scoring, the codes were broken, and the data compiled.

Colitis models: For the DSS colitis model, animals were provided with drinking water containing 2.5-3% DSS (#0216011010. MP Biomedicals, Santa Ana, CA) for 7 days. Mice were injected with 100 μg of anti-CTLA-4 mAb (#BE0164, clone 9D9, Bio X Cell, West Lebanon, NH) or isotype control (#BE0086, MCP-11, Bio X Cell) twice (3 and 1 day before DSS administration). For the T-cell transfer induced colitis model, isolated splenic T cells from WT B6 and Prf^{-/-}mice were stained with DAPI (#422801, 1 μM, Biolenged), APC-Cy7 anti-CD4 (#560246, GK1.5, 1:100, BD Biosciences, San Jose, CA), APC anti-CD25 (#101910, 3C7, 1:100, Biolegend), FITC anti-CD44 (#103006, IM7, 1:100, Biolegend) and PE anti-CD45RB (#103308, C363-16A, 1:100, Biolegend). CD4⁺CD25⁻CD44⁻CD45RB^{hi} cells were sorted with the MoFlo Astrios cell sorter (Beckman Coulter, Indianapolis, IN) and intraperitoneally injected into Rag-1^{-/-}recipients.

Liquid chromatography mass spectrometry (LC/MS): To quantitate tricarboxylic acid cycle metabolites in vivo, samples (colon, ileum and kidney) from mice 7 and 21 days post HCT were harvested, homogenized, and snap-frozen in liquid N₂. Tricarboxylic acid metabolites were extracted from tissue homogenates with a mixture of methanol, chloroform, and water (8:1:1) containing C13 isotope-labeled internal standards for citrate, succinate, fumarate, malate, alpha-ketoglutarate, lactate and pyruvate. Liquid chromatography-mass spectrometry (LC/MS) analysis was performed on an Agilent system consisting of a 1260 UPLC module coupled with a 6410 Triple Quadrupole (QQQ) mass spectrometer (Agilent Technologies, Santa Clara, CA). Data were processed using MassHunter Quantitative analysis version B.07.00. Metabolites were normalized to the nearest isotope-labeled internal standard and quantitated using a linear calibration curve^{66,67}.The tissue levels were normalized by the protein concentration of the homogenized tissues.

Acyl-carnitine quantitation: Tissues were subjected to targeted metabolomics analysis by LC/MS for determination of acyl-carnitines as previously described⁶⁸. Briefly, samples were homogenized in 25 mM phosphate buffer (pH 4.9) and extracted with cold 2:1:1 isopropanol: acetonitrile: methanol. Known amounts of isotope-labeled carnitines were used as internal standards and analyzed for LC/ESI/MS/MS analysis, an Agilent 6410 triple quadruple MS system equipped with an Agilent 1200 LC system and electrospray ionization (ESI) source was utilized and were detected in the multiple reaction monitoring (MRM) mode and relative peak areas were obtained.

Isotope tracing assay assessing label incorporation into TCA cycle metabolite in vivo: Animals 7 and 21 days post HCT were intragastrically gavaged with a bolus (2 g/kg) of either labeled ¹³C-glucose (#CLM-1396-1, Cambridge Isotopes, Tewksbury, MA) or non-labeled ¹²C-glucose after 9h fasting. The small and large intestinal epithelial cells were then isolated 4 hours later and prepared for analysis as above. The incorporation of ¹³C-glucose into the TCA cycle metabolites in the intestinal tissue was measured using LC/MS performed on Agilent 6520 high resolution Q-TOF (quadrupole-time of flight instrument) coupled with an Agilent 1200 HPLC system (Agilent Technologies, New Castle, DE), equipped with an electrospray source. The extract was subjected to hydrophilic interaction chromatography using Phenomenex Luna NH₂ column (particle size 3 μm; 1×150 mm) at a flow rate of 0.07 mL/min. Solvent A was 5 mM ammonium acetate with pH 9.9 and solvent B was acetonitrile. The column was equilibrated with 80% solvent B. The gradient was: 20-100% solvent A over 15 min; 100% solvent A over 5 min; 20% solvent B for 0.1 min; and 20% solvent A for 15.9 min. Liquid chromatography electrospray ionization (LC/ESI) MS in the negative mode was performed by the Q-TOF instrument using the following parameters: spray voltage 3000 V, drying gas flow 10 L/min, drying gas temperature 350° C., and nebulizer pressure 20 psi. Fragmentor voltage was 150 V in full scan mode. Mass range between m/z 100 to 1500 was scanned to obtain full scan mass spectra. Two reference masses at m/z121.050873 and m/z 922.009798 were used to obtain accurate mass measurement within 5 ppm. All chromatograms and corresponding spectra of TCA metabolites: citrate, succinate and malate and their corresponding ¹³C labeled counterparts were extracted and deconvoluted using MassHunter software (Agilent Technologies, New Castle, DE). Retention time consistency was manually rechecked and compared to authentic compounds that were injected under similar chromatographic conditions. For tissue extracts, metabolite concentrations were normalized to protein content, which was determined by the Bradford-Lowry method. For the isotope tracing analysis, peak areas of the labeled compounds were normalized to natural abundance of the label and represented as ratios to the total compound peak area.

¹³C-glucose or ¹³C-glutamine (#CLM-1822, Cambridge Isotopes) tracing in vitro was performed using glucose-free DMEM (#A1443001, Thermo Fisher Scientific, Waltham, MA) supplemented with 1 mM sodium pyruvate (#11360070, Thermo Fisher Scientific), 1% HEPES (#15630080, Thermo Fisher Scientific), 2% BSA (#126575, Sigma-Aldrich), 10 uM Y-27632 (#Y0503, Sigma-Aldrich) and either 17.5 mM ¹²C or ¹³C-glucose, and either 2 mM ¹²C or ¹³C-glutamine. Isolated intestinal epithelial cells (IECs) from naïve, syngeneic and allogeneic animals 7 and 21 days post HCT were cultured for 2 hours in ¹²C or ¹³C glucose/glutamine labeling media. Intracellular metabolite fractions were prepared from cells that were lysed with cold (−80° C.) 80% methanol, then clarified by centrifugation. Metabolite pellets from intracellular fractions were normalized to the protein content of a parallel sample, and all samples were lyophilized via speed vac. Dried metabolite pellets from cells were re-suspended in 35 μL 50:50 MeOH: H₂O mixture for metabolomics analysis. Agilent 1260 UHPLC combined with a 6520 Accurate-Mass Q-TOF LC/MS was utilized. Agilent MassHunter Workstation Software LC/MS Data Acquisition for 6200 series TOF/6500 series QTOF (B.06.01) was used for calibration and data acquisition. A Waters Acquity UPLC BEH amide column (2.1×100 mm, 1.7 μm) was used with mobile phase (A) consisting of 20 mM NH₄OAc in water pH 9.6, and mobile phase (B) consisting of acetonitrile. The following gradient was used: mobile phase (B) was held at 85% for 1 min, increased to 65% at 12 min, then to 40% at 15 min and held for 5 min before going to the initial condition and holding for 10 min. The column was cooled to 40° C. and 3 μL of sample was injected into the LC-MS with a flow rate of 0.2 mL/min. Calibration of TOF MS was achieved using the Agilent ESI Low Concentration Tuning Mix. Key parameters for both acquisition modes were: mass range 100-1200 da, Gas temp 350° C., Fragmentor 150 V, Skimmer 65 V, Drying Gas 10 L/min, Nebulizer at 20 psi, Vcap 3500 V and Ref Nebulizer at 20 psi. For the negative mode the reference ions were at 119.0363 and 980.01637 m/z whereas for positive acquisition mode, reference ions at 121.050873 and 959.9657 m/z. For data analysis, we used Agilent MassHunter Workstation Software Profinder B.08.00 with Batch Targeted Feature Extraction and Batch Isotopologue Extraction and Qualitative Analysis B.07.00. Various parameter combinations, e.g., mass and RT tolerance, were used to find the best peaks and signals by manual inspection. Key parameters were: mass tolerance=20 or 10 ppm and RT tolerance=1 or 0.5 min. Isotopologue ion thresholds and the anchor ion height threshold were set to 250 counts and the threshold of the sum of ion heights to 500 counts. Coelution correlation threshold was set to 0.3⁶⁹.

Cell isolation: Luminal contents from dissected colon and ileum were flushed with CMF buffer: Ca²⁺/Mg²⁺free HBSS (#14185052, Thermo Fisher Scientific) supplemented with 25 mM sodium bicarbonate (#S6014, Sigma-Aldrich) and 2% FBS (#100-106, Gemini Bio Products, USA). Intestines were then minced into 5 mm pieces, washed with CMF buffer four times, transferred to CMF with 5 mM EDTA (#51201, Lonza, USA), and incubated at 37° C. for 40 minutes (shaking tubes every 10 minutes). Supernatants containing IECs were then transferred through 100 μM cell filter followed by incubation on ice for 10 minutes to allow sedimentation. Supernatants were again transferred through a 75 μM cell filter. CD326⁺IECs were next purified with APC-anti-CD326 (G8.8, #118214, 1:200, Biolegend) and anti-APC magnetic microbeads (#130-090-855, Miltenyi Biotec).

Succinate dehydrogenase functional staining: Specimens were dissected from naïve, syngeneic and allogeneic WT B6 mice at 7 and 21 days post HCT. The tissues were rinsed in ice cold PBS and were then frozen in 2-methylbutane (#M32631, Sigma-Aldrich) and dry ice. Frozen sections (cut at 8 μm thick) were directly placed in SDH solution; 48 mM disodium succinate hexahydrate (#8201510100, Sigma-Aldrich), 0.75 mM sodium azide (#S8032, Sigma-Aldrich), 0.5 mM disodium EDTA (#324503, Sigma-Aldrich), 13 mM sodium phosphate monobasic monohydrate (#S3522, Sigma-Aldrich), 87 mM sodium phosphate dibasic heptahydrate (#S9390, Sigma-Aldrich), 998.9 μM 1-methoxy-5-methylphenazinium methyl sulfate (M8640, Sigma-Aldrich), 1.5 mM nitroblue tetrazolium (#484235, Sigma-Aldrich) at 37° C. for 30 minutes and then the slides were rinsed in ddH₂O. After mounting the slides, images were obtained using Olympus BX51M (Olympus, Japan) and the SDH activity was quantified using Image J (National Institutes of Health, Bethesda, MA, USA).

Mitochondria Isolation: Isolated IECs from syngeneic and allogeneic recipients in mitochondria isolation buffer (MIB) were disrupted using a glass homogenizer with 2-3 strokes. MIB was composed of 70 mM sucrose (#S0389, Sigma-Aldrich), 210 mM mannitol (#M9546, Sigma-Aldrich), 5 mM HEPES, 1 mM EGTA (#E0396, Sigma-Aldrich), 0.5% BSA, pH 7.2 and protease inhibitor cocktail (#78429, Sigma-Aldrich). Homogenate was centrifuged at 800 g for 10 min at 4° C. Following centrifugation, fat/lipid was carefully aspirated, and the remaining supernatant was decanted through 2 layers of cheesecloth to a separate tube and centrifuged twice at 8000 g for 15 min at 4° C. After removal of the light mitochondrial layer, the pellet was resuspended in MIB and centrifuged at 8500 g for 10 min. The final pellet was resuspended in a minimal volume of mitochondrial assay solution (MAS) and kept on ice. MAS comprises 70 mM sucrose, 220 mM mannitol, 10 mM potassium monobasic phosphate (#P5655, Sigma-Aldrich), 5mM magnesium chloride (#M8266, Sigma-Aldrich), 2 mM HEPES, 1 mM EGTA, 0.2% BSA, and protease inhibitor cocktail at the pH of 7.2. Total protein (mg/ml) was determined via Pierce BCA Protein Assay (#23225, Thermo Scientific).

Immunoblot analysis: Isolated mitochondria or IECs were lysed in RIPA buffer (#89901, Thermo Scientific). Equal amounts of proteins were loaded on 4-12% SDS-PAGE gel (#NP0321, Thermo Scientific), electrophoresed and subsequently transferred to a PVDF membrane (#ISEQ85R, Sigma-Aldrich) using a Bio-Rad semi-dry transfer cell (20 V, 1 h). Blots were incubated with anti-SDHA (#ab14715, 2E3GC12FB2AE2, 0.1 μg/ml, Abcam, Cambridge, UK), Total OXPHOS rodent antibody cocktail (#ab110413, 1:250, Abcam), Tom20 (#42406, D8T4N, 1:1000, Cell Signaling Technology, Danvers, MA), SUNCR1 (LS-B15693, 1:500, LSBio) and anti-β actin (#8226, mAbcam8226, 1:3000, Abcam) primary antibodies overnight at 4° C. Incubation with secondary anti-rabbit-HRP (#7074S, Cell Signaling Technology), anti-mouse-HRP (#sc-516102, Santa Cruz Biotechnology, Dallas, TX) was performed at room temperature for 1 hour. Bound antibody was detected using Super Signal ECL substrate (Thermo Scientific) and quantitated using ChemiDoc MP Imaging system (BioRad, Hercules, CA). Densitometric analysis was performed using Image J.

SDHA cleavage assay: 75 ug Isolated mitochondria in 25 ul EDB buffer (10 mM HEPES, 50 mM NaCl, 2 mM MgCl₂, 5 mM EGTA and 1 mM DTT) were incubated with 75 ng mouse Granzyme B at 37 degrees and then separated by SDS-PAGE⁷⁰. Mouse recombinant SDHA protein with His Tag (LS-G26030, LSBio, Seattle, WA) and mouse recombinant SDHB protein with His Tag (LS-G26026, LSBio) in EDB buffer (25 ug in 8.3 ul) were incubated with 25 ng mouse Granzyme B at 37 degrees for 90 minutes. Proteins were then separated by SDS-PAGE and blotted with anti-His Tag antibody (LS-C60077, 1:1000, LSBio) and anti-SDHA (#ab139181, 1:1000, Abcam).

BN-PAGE: Isolated mitochondria was lysed in NativePAGE sample buffer (#BN2008, Thermo Scientific) containing 2% digitonin. 50 ug of total protein was loaded on 3-12% NativePAGE gel (#BN1001, Thermo Scientific). Gels were stained by Colloidal Blue Staining Kit (#LC6025, Thermo Scientific). For the second dimension, SDS-PAGE, gel lanes from BN gels were placed onto top of a 4-12% NuPage 2D well (#NP0326, Thermo Scientific). After separation and blotting, membranes were probed with Total OXPHOS blue native antibody cocktail (#ab110412, 1:250, Abcam).

Fluorescent immunohistochemistry staining: Sections were prepared as mentioned in the SDH functional staining method. The sections were fixed in 4% paraformaldehyde (#P6148, Sigma-Aldrich) for 30 minutes. Then, the sections were washed 3 times in PBS and permeabilized in PBS containing 0.3% Triton-X100 (#X100, Sigma-Aldrich) for 2 hours. Permeabilized sections were blocked for 2 hours (10% normal goat serum in PBS containing 0.15% Triton X-100) and incubated with primary antibodies overnight at 4° C. The following primary antibodies were used; anti-SDHA (#ab14715, 2E3GC12FB2AE2, 1:100, Abcam), COX IV (#ab16056, 20E8C12, 1:200, Abcam), Granzyme B (#NB100-684, 1:100, Novus Biologicals, Centennial, CO) overnight at 4. Incubation with secondary Cy3 anti-mouse-IgG (#405309, Poly4053, 1:250, BioLegend, San Diego, CA), Cy3 anti-rabbit-IgG (#406402, Poly4064, 1:250, BioLegend) and Dy-Light488 anti-rabbit-IgG (#406404, Poly4064, 1:250, BioLegend) was performed at room temperature for 1 hour. Slides were mounted with coverslips using Prolong Gold with DAPI (#P36931, Thermo Scientific). For human samples, formalin fixed paraffin embedded specimens from patients were cut to a thickness of 5 μm. Heat-induced antigen retrieval was performed with 10 mM sodium citrate buffer followed by staining with primary antibodies. Slides were imaged using a Nikon A1confocal microscope (Nikon, Melville, NY). SDH levels were quantified and measured in intensity from Cy3 positive epithelial cells using Image J. For primary mice colonic epithelial cells, cells were spun down and put on coverslips. After 10 minutes of incubation at 37° C., cells were fixed with 3% paraformaldehyde for 20 minutes, quenched with 100 mM Glycine (#G8998, Sigma-Aldrich) 3 times for 3 minutes and permeabilized in PBS containing 0.3% Triton-X100 for 1 minute. Then, the cells were blocked, stained with primary and secondary antibodies, and mounted.

Transmission Electron Microscopy and immunogold immunohistochemistry: Colon and ileum were harvested, diced into lmm cubes and fixed with 4% formaldehyde and 2.5% glutaraldehyde (#G5882, Sigma-Aldrich) in 0.1 M phosphate buffer (PB, pH 7.4) overnight. Next, the tissues were immersed in 0.2 M sucrose and 0.1 M PB, and then post-fixed in 1% osmium tetroxide (#75632, Sigma-Aldrich) in 0.1 M PB. Then, the tissue was dehydrated in ascending concentrations of ethanol, treated with propylene oxide, and embedded in Epon epoxy resin. Semi-thin sections were stained with toluidine blue for tissue identification. Regions of interest were selected, cut into ultra-thin section (70 nm thick) mounted on copper grids, and then stained with uranyl acetate and lead citrate. For immunogold immunohistochemistry, sections were blocked with blocking solution for goat gold conjugates (#905.002, AURION, Netherland) for 2 hours and then incubated with anti-SDHA antibody (1:10) overnight at 4° C. After rinsing, sections were incubated with 10 nm gold particle conjugated anti-mouse IgG (H&L) (#810.022, 1:50, AURION) for 2 hours at room temperature and then fixed with 2.5% glutaraldehyde for 10 min before staining with 4% uranyl acetate for 15 minutes. The samples were examined using a JEM-1400 electron microscope (JEOL, Peabody, MA) at 80 kV. Images were recorded digitally using a XR401 camera system operated with AMT software (Advanced Microscopy Techniques Corp., Danvers, MA). Gold particles in the mitochondria were quantified in a blinded manner by L.L. After quantification, the codes were broken, and the data were compiled.

MtDNA Copy Number Amtiysis: Total DNA was extracted from isolated IECs using the DNeasy Blood & Tissue Kit (#69504, QIAGEN, Germantown, MD). PowerUP SYBR green master mix (#A25742, Applied Biosystems, Foster City, CA) and the following primers were used; 5′-CAAACACTTATTACAACCCAAGAACA-3′ (SEQ ID NO.: 1) and 5′-TCATATTATGGCTATGGGTCAGG-3′ (SEQ ID NO.: 2) (ND1; NADH:ubiquinone oxidoreductase core subunit 1); 5′-AATCTACCATCCTCCGTGAAACC-3′(SEQ ID NO.: 3) and 5′-TCAGTTTAGCTACCCCCAAGTTTAA-3′(SEQ ID NO.: 4) (D-loop1; displacement loop1); 5′- CCCTTCCCCATTTGGTCT-3′(SEQ ID NO.: 5) and 5′-TGGTTTCACGGAGGATGG-3′(SEQ ID NO.: 6) (D-loop2)⁷¹; 5′-TCCTCCGTGAAACCAACAA-3′(SEQ ID NO.: 7) and 5′-AGCGAGAAGAGGGGCATA-3′(SEQ ID NO.: 8) (D-loop3); 5′- GCTTTCCACTTCATCTTACCATTTA-3′(SEQ ID NO.: 9) and 5′-TGTTGGGTTGTTTGATCCTG-3′(SEQ ID NO.: 10) (CytB: cytochrome B); 5′-CACTGCCTGCCCAGTGA-3′(SEQ ID NO.: 11) and 5′-ATACCGCGGCCGTTAAA-3′(SEQ ID NO.: 12) (16S); 5′-AACGGATCCACAGCCGTA-3′(SEQ ID NO.: 13) and 5′-AGTCCTCGGGCCATGATT-3′(SEQ ID NO.: 14) (ND4); and 5′-GCTTAATTTGACTCAACACGGGA-3′(SEQ ID NO.: 15) and 5′-AGCTATCAATCTGTCAATCCTGTC-3′(SEQ ID NO.: 16) (18S). All primers were verified for the production of a single specific PCR product via melting curve analysis.

Succinate quantification: Isolated IECs were collected, processed and analysed using the Succinate Assay Kit (#ab204718, Abcam) according to the manufacturer's protocol.

Seahorse analysis: Analysis was performed on IECs after HCT. Cells were washed three times in complete seahorse XF assay medium (#103335-100, Aglient technologies, Santa Clara, CA) with 17.5 mM glucose, 1 mM sodium pyruvate, 2 mM glutamine, 2% BSA 10 uM Y-27632 and 1% penicillin-streptomycin (#516106, Sigma-Aldrich) adjusted to pH 7.4. Cells were plated at 8×10⁴ cells per well in a Seahorse assay plate, pretreated with matrigel (#354262, Corning, Corning, NY). Cells were equilibrated to 37° C. for 30 min before assay. Respiration profile was assessed in 96XF instrument with Mitostress assay as indicated upon cell treatment with oligomycin (#75351, 2 μM, Sigma-Aldrich), FCCP (#C2990, 1 μM, Sigma-Aldrich), rotenone (#R8875, 0.5 μM, Sigma-Aldrich) and antimycin A (#A8674, 0.5 μM, Sigma-Aldrich)¹⁴.

ATP detection assay: Isolated mitochondria from IECs were incubated in MAS supplemented with 5 mM ADP (#A2754, Sigma-Aldrich), 10 mM succinic acid (#S3674, Sigma-Aldrich) and 2 μM rotenone on white walled 96-well plates (#3917, Corning) at 8×10⁴ cells per well in triplicate for 2 hours. The ATP level was measured using the Cell Titer Glo 2.0 luminescence assay (#G9243, Promega, Madison, WI). Luminescence was measured for 500 ms using a SpectraMax M3 Microplate reader (Molecular Devices, San Jose, CA).

Complex I enzyme activity assay: Isolated mitochondria from IECs were loaded on 96-well plates at 5 μg per well in triplicate. Enzyme activity was measured using the colorimetric Complex I Enzyme Activity Assay Kit (#ab109721, Abcam) following manufacturer's instructions.

NAD⁺/Nicotinamide, Adenine Dinuckotide Diaphorase Assay: Total intracellular NAD (NADH+NAD⁺) and nicotinamide adenine dinucleotide diaphorase (NADH) levels in IECs were measured using the NAD⁺/NADH Quantification Kit (#K337-100, BioVision, Milpitas, CA) following the manufacturer's recommendations The NAD⁺/NADH ratio was then calculated based on levels of NAD and NADH.

⁵¹Cr release assay: Splenic T cells from WT-B6 and BALB/c animals and irradiated (30Gy) red blood cell lysed splenocytes from WT-B6 mice were co-cultured in 5:2 ratio for 96 hours. Splenic CD8⁺T cells were isolated from above co-cultured cells, purified with a CD8⁺T-cell isolation kit (#130-117-044, Miltenyi Biotec) and used as effector cells. PCECs were pre-treated with 5mM DMM, 0.5mM DI, 10nM atpenin A5 or vehicle for 3 hours. 2×10⁶ treated cells were incubated with 2 MBq of Na₂⁵¹CrO₄ (#NEZ030001MC, PerkinElmer, Waltham, MA) for 2 hours at 37° C. in a 5% CO₂ atmosphere and were used as target cells. After washing, 4×10³ labeled targets were resuspended and added to triplicate wells at varying effector-to-target ratios and then incubated for 4 h. Maximal and minimum release was determined by the addition of Triton-X or media alone to targets, respectively.

For the transwell assays, 2×10⁵ labeled targets were placed on the bottom chamber and effector cells were on the bottom or on the 0.4 uM transwell membrane for 12 hours at 37° C. in 5% CO₂. Cells were incubated in fresh cell culture medium or conditioined from supernatant from effector cells. After incubation, supernatants were transferred to a Luma plate (#600633, PerkinElmer) and ⁵¹Cr activity was determined using Top Count NXT (Hewlett Packard, Palo Alto, CA).

Flow cytometry: Cells were re-suspended in 2% BSA in PBS and stained with the following antibodies and reagents; FITC-anti-CD326 (G8.8, #118208, 1:200), APC-anti-CD326 (G8.8, #118214, 1:200), DAPI, CellROX Deep Red (#C10491, 500 nM, Thermo Scientific), MitoSOX (#M36008, 5 μM, Thermo Scientific), JC-1 (#M34152, 2 μM, Thermo Scientific), 7-AAD (#420404, 1:100, BioLegend), Annexin V (#640947, 1:20, Biolegend), anti-CD16/CD32 antibody (BD Biosciences, 2.4G2), APC-anti-CD45.2 (#109814, 1:200, BioLegend), and CellTracker Green CMFDA Dye (#C7025, 1 μM, Invitrogen) according to manufacturer's protocol. Cells were run on Attune NxT flow cytometer (Thermo Scientific) and analyzed using FlowJo v10.2 (FlowJo LLC, Ashland, OR).

RNA isolation and RT-PCR: Total RNA from single-cell suspensions was isolated using the RNeasy Kit (#74104, QIAGEN, Hilden, Germany) and reverse transcribed into cDNA using the High Capacity cDNA Reverse Transcription Kit (#4374966, Applied Biosystems). The following primers and PowerUP SYBR green polymerase were used to detect the following transcripts: 5′-ACATGCAGAAGTCGATGCAG-3′(SEQ ID NO.: 17) and 5′-CATTCCCCTGTCGAATGTCT-3′(SEQ ID NO.: 18) (Sdha); and 5′-TGACCTCAACTACATGGTCTACA-3′(SEQ ID NO.: 19) and 5′-CTTCCCATTCTCGGCCTTG-3′(SEQ ID NO.: 20) (Gapdh). All reactions were performed according to manufacturer's instructions. All primers were verified for the production of a single specific PCR product viamelting curve analysis.

Transcriptome analysis of colonic epithelial cells from bone marrow transplant recipient mice: C57BL/B6 mice used as recipients. Donor mice were C57BL/B6 for syngeneic group and BALB/c for allogeneic group. Recipient mice were received 1000cGy TBI on day −1 and were injected 3×10⁶ CD90.2+T cells and 5×10⁶ bone marrow cells on day 0. Colonic epithelial cells were isolated from recipient mice at 7 days after HCT day 7 and processed for RNA isolation. Total RNA from colonic epithelial cells was isolated using the RNeasy Kit (#74104, QIAGEN, Hilden, Germany). Library preparations and sequencing reactions were conducted at GENEWIZ, LLC. (South Plainfield, NJ, USA). RNA samples were quantified using Qubit 4.0 Fluorometer (Invitrogen, Carlsbad, CA, USA) and RNA integrity was checked using Agilent TapeStation 4200 (Agilent Technologies, Palo Alto, CA, USA). RNA sequencing libraries were prepared using the NEBNext Ultra II RNA Library Prep Kit for Illumina following manufacturer's instructions (NEB, Ipswich, MA, USA). Briefly, mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified using MiSeq Nano or Qubit 4.0 Fluorometer (Invitrogen, Carlsbad, CA) as well as by quantitative PCR (KAPA Biosystems, Wilmington, MA, USA). The sequencing libraries were pooled and clustered on one lane of a flowcell. After clustering, the flowcell was loaded on the Illumina HiSeq instrument (4000) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. Raw Illumina reads for each sample were mapped to the Mouse genome (mm10) using STAR (version 2.5.2a) with the—quantMode GeneCounts option in order to directly generate a matrix of counts for each gene. R (version 3.6.1) package DESeq2 was used to determine statistical significance of gene expression differences between Allogeneic and Syngeneic samples. Pathway analysis was performed using PantherDB⁷², where all genes from DESeq2 with a multiple hypothesis corrected p-value below 0.05 were input into the PantherDB web portal, analyzed against all the mouse genes in the database using the Fisher's exact test with False Discovery Rate (FDR) correction and using the PANTHER GO-slim Biological Processes Annotation Data set.). All original data were deposited in the NCBI's GEO database (GSE158259).

Generation of normal SDHA protein and mutated SDHA protein: Target DNA sequence of mouse normal SDHA and mutated SDHA was optimized and synthesized (GenScript). The synthesized sequence was cloned into vector pET-30a(+) with His tag for protein expression in E. coli. E. coli strain BL21 Star™(DE3) was transformed with recombinant plasmid. A single colony was inoculated into LB medium containing related antibiotic; culture was incubated in 37° C. at 200 rpm and then induced with IPTG. SDS-PAGE was used to monitor the expression. Recombinant BL21 Star™ (DE3) stored in glycerol was inoculated into TB medium containing related antibiotic and cultured at 37° C. When the OD600 reached about 1.2, cell culture was induced with IPTG at 15° C./16 h. Cells were harvested by centrifugation. Cell pellets were resuspended with lysis buffer followed by sonication. The precipitate after centrifugation was dissolved using denaturing agent. Target protein was obtained by one-step purification using Ni column. Target protein was sterilized by 0.22 μm filter before stored in aliquots. The concentration was determined by Bradford protein assay with BSA as standard. The protein purity and molecular weight were determined by standard SDS-PAGE along with Western blot confirmation. Normal SDHA protein with His and mutated SDHA protein with His Tag in PBS (10 ug in 10 ul) were incubated with 50 ng mouse Granzyme B at 37 degrees for 6 hours. Proteins were then separated by SDS-PAGE and blotted with anti-His Tag antibody (LS-C60077, 1:1000, LSBio).

Human Subjects and Data: Colonic biopsy samples embedded in paraffin with deidentified and blinded patient data diagnosed as GVHD or non-GVHD from the University of Michigan Department of Pathology was provided without any information. All protocols and procedures for the human studies were approved by the university Institutional Review Board.

Statistical analysis: Experiments were conducted with technical and biological replicates at an appropriate sample size, as estimated by our prior experience. No statistical methods were used to predetermine sample size. No methods of randomization and no blinding were applied. All data were replicated independently at least once as indicated in the figure legends, and all attempts to reproduce experimental data were successful. Bars and error bars represent the mean and s.e.m., respectively. All statistical analysis was performed using Graph Pad Prism 7 (Graph Pad Software Inc, San Diego, CA). P values<0.05 were considered as significant: *P<0.05, **P<0.01, ***P<0.001, ****P<0.0001; P values>0.05 were considered as non-significant. All sample sizes and statistical tests used are detailed in each figure legend.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### Incorporation By Reference

The entire disclosure of each of the patent documents and scientific articles referred to herein is incorporated by reference for all purposes. Specifically, the following references denoted herein are incorporated by reference for all purposes:
1 Strober, W., Fuss, I. & Mannon, P. The fundamental basis of inflammatory bowel disease. J Clin Invest 117, 514-521, doi:10.1172/jci30587 (2007).
2 Yilmaz, B. et al. Microbial network disturbances in relapsing refractory Crohn's disease. Nat Med 25, 323-336, doi:10.1038/s41591-018-0308-z (2019).
3 Cleynen, I. et al. Inherited determinants of Crohn's disease and ulcerative colitis phenotypes: a genetic association study. Lancet 387, 156-167, doi:10.1016/s0140-6736(15)00465-1 (2016).
4 Jostins, L. et al. Host-microbe interactions have shaped the genetic architecture of inflammatory bowel disease. Nature 491, 119-124, doi:10.1038/nature11582 (2012).
5 Martins, F. et al. Adverse effects of immune-checkpoint inhibitors: epidemiology, management and surveillance. Nat Rev Clin Oncol, doi:10.1038/s41571-019-0218-0 (2019).
6 Ferrara, J. L., Levine, J. E., Reddy, P. & Holler, E. Graft-versus-host disease. Lancet 373, 1550-1561, doi:10.1016/s0140-6736(09)60237-3 (2009).
7 Neurath, M. F. Targeting immune cell circuits and trafficking in inflammatory bowel disease. Nat Immunol, doi:10.1038/s41590-019-0415-0 (2019).
8 Zeiser, R. & Blazar, B. R. Acute Graft-versus-Host Disease-Biologic Process, Prevention, and Therapy. N Engl J Med 377, 2167-2179, doi:10.1056/NEJMra1609337 (2017).
9 O'Neill, L. A. & Pearce, E. J. Immunometabolism governs dendritic cell and macrophage function. J Exp Med 213, 15-23, doi:10.1084/jem.20151570 (2016).
10 Everts, B. et al. TLR-driven early glycolytic reprogramming via the kinases TBK1-IKKvarepsilon supports the anabolic demands of dendritic cell activation. Nat Immunol 15, 323-332, doi:10.1038/ni.2833 (2014).
11 Buck, M. D. et al. Mitochondrial Dynamics Controls T Cell Fate through Metabolic Programming. Cell 166, 63-76, doi:10.1016/j.cell.2016.05.035 (2016).
12 Albenberg, L. et al. Correlation between intraluminal oxygen gradient and radial partitioning of intestinal microbiota. Gastroenterology 147, 1055-1063.e1058, doi:10.1053/j.gastro.2014.07.020 (2014).
13 He, G. et al. Noninvasive measurement of anatomic structure and intraluminal oxygenation in the gastrointestinal tract of living mice with spatial and spectral EPR imaging. Proc Natl Acad Sci USA 96, 4586-4591, doi:10.1073/pnas.96.8.4586 (1999).
14 Fan, Y. Y. et al. A bioassay to measure energy metabolism in mouse colonic crypts, organoids, and sorted stem cells. Am J Physiol Gastrointest Liver Physiol 309, G1-9, doi:10.1152/ajpgi.00052.2015 (2015).
15 Donohoe, D. R. et al. The microbiome and butyrate regulate energy metabolism and autophagy in the mammalian colon. Cell Metab 13, 517-526, doi:10.1016/j.cmet.2011.02.018 (2011).
16 Roediger, W. E. Role of anaerobic bacteria in the metabolic welfare of the colonic mucosa in man. Gut 21, 793-798, doi:10.1136/gut.21.9.793 (1980).
17 Cogliati, S., Enriquez, J. A. & Scorrano, L. Mitochondrial Cristae: Where Beauty Meets Functionality. Trends Biochem Sci 41, 261-273, doi:10.1016/j.tibs.2016.01.001 (2016).
18 Weber, J. S., Dummer, R., de Pril, V., Lebbe, C. & Hodi, F. S. Patterns of onset and resolution of immune-related adverse events of special interest with ipilimumab: detailed safety analysis from a phase 3 trial in patients with advanced melanoma. Cancer 119, 1675-1682, doi:10.1002/cncr.27969 (2013).
19 Wang, F., Yin, Q., Chen, L. & Davis, M. M. Bifidobacterium can mitigate intestinal immunopathology in the context of CTLA-4 blockade. Proc Natl Acad Sci USA 115, 157-161, doi:10.1073/pnas.1712901115 (2018).
20 Perez-Ruiz, E. et al. Prophylactic TNF blockade uncouples efficacy and toxicity in dual CTLA-4 and PD-1 immunotherapy. Nature 569, 428-432, doi:10.1038/s41586-019-1162-y (2019).
21 Mills, E. L. et al. Succinate Dehydrogenase Supports Metabolic Repurposing of Mitochondria to Drive Inflammatory Macrophages. Cell 167, 457-470.e413, doi:10.1016/j.cell.2016.08.064 (2016).
22 Bailis, W. et al. Distinct modes of mitochondrial metabolism uncouple T cell differentiation and function. Nature, doi:10.1038/s41586-019-1311-3 (2019).
23 Dotiwala, F. et al. Killer lymphocytes use granulysin, perforin and granzymes to kill intracellular parasites. Nat Med 22, 210-216, doi:10.1038/nm.4023 (2016).
24 Luan, H. H. et al. GDF15 Is an Inflammation-Induced Central Mediator of Tissue Tolerance. Cell, doi:10.1016/j.ce11.2019.07.033 (2019).
25 Tannahill, G. M. et al. Succinate is an inflammatory signal that induces IL-1beta through HIF-1alpha. Nature 496, 238-242, doi:10.1038/nature11986 (2013).
26 Littlewood-Evans, A. et al. GPR91 senses extracellular succinate released from inflammatory macrophages and exacerbates rheumatoid arthritis. J Exp Med 213, 1655-1662, doi:10.1084/jem.20160061 (2016).
27 Ghezzi, D. et al. SDHAF1, encoding a LYR complex-II specific assembly factor, is mutated in SDH-defective infantile leukoencephalopathy. Nat Genet 41, 654-656, doi:10.1038/ng.378 (2009).
28 Na, U. et al. The LYR factors SDHAF1 and SDHAF3 mediate maturation of the iron-sulfur subunit of succinate dehydrogenase. Cell Metab 20, 253-266, doi:10.1016/j.cmet.2014.05.014 (2014).
29 Michelucci, A. et al. Immune-responsive gene 1 protein links metabolism to immunity by catalyzing itaconic acid production. Proc Natl Acad Sci USA 110, 7820-7825, doi:10.1073/pnas.1218599110 (2013).
30 Harris, J. L., Peterson, E. P., Hudig, D., Thornberry, N. A. & Craik, C. S. Definition and redesign of the extended substrate specificity of granzyme B. J Biol Chem 273, 27364-27373, doi:10.1074/jbc.273.42.27364 (1998).
31 Sipione, S. et al. Identification of a novel human granzyme B inhibitor secreted by cultured sertoli cells. J Immunol 177, 5051-5058, doi:10.4049/jimmunol.177.8.5051 (2006).
32 Wu, S. R. & Reddy, P. Tissue tolerance: a distinct concept to control acute GVHD severity. Blood 129, 1747-1752, doi:10.1182/blood-2016-09-740431 (2017).
33 Mehta, M. M., Weinberg, S. E. & Chandel, N. S. Mitochondrial control of immunity: beyond ATP. Nat Rev Immunol 17, 608-620, doi:10.1038/nri.2017.66 (2017).
34 Vander Heiden, M. G., Cantley, L. C. & Thompson, C. B. Understanding the Warburg effect: the metabolic requirements of cell proliferation. Science 324, 1029-1033, doi:10.1126/science.1160809 (2009).
35 Chapman, N. M., Boothby, M. R. & Chi, H. Metabolic coordination of T cell quiescence and activation. Nat Rev Immunol, doi:10.1038/s41577-019-0203-y (2019).
36 Sugiura, A. & Rathmell, J. C. Metabolic Barriers to T Cell Function in Tumors. J Immunol 200, 400-407, doi:10.4049/jimmunol.1701041 (2018).
37 Chouchani, E. T. et al. Ischaemic accumulation of succinate controls reperfusion injury through mitochondrial ROS. Nature 515, 431-435, doi:10.1038/nature13909 (2014).
38 Caruso, R., Warner, N., Inohara, N. & Nunez, G. NOD1 and NOD2: signaling, host defense, and inflammatory disease. Immunity 41, 898-908, doi:10.1016/j.immuni.2014.12.010 (2014).
39 Jacquemin, G. et al. Granzyme B-induced mitochondrial ROS are required for apoptosis. Cell Death Differ 22, 862-874, doi:10.1038/cdd.2014.180 (2015).
40 Martinvalet, D., Zhu, P. & Lieberman, J. Granzyme A induces caspase-independent mitochondrial damage, a required first step for apoptosis. Immunity 22, 355-370, doi:10.1016/j.immuni.2005.02.004 (2005).
41 Dotiwala, F. et al. Granzyme B Disrupts Central Metabolism and Protein Synthesis in Bacteria to Promote an Immune Cell Death Program. Cell 171, 1125-1137 e1111, doi:10.1016/j.cell.2017.10.004 (2017).
42 Walch, M. et al. Cytotoxic Cells Kill Intracellular Bacteria through Granulysin-Mediated Delivery of Granzymes. Cell 161, 1229, doi:10.1016/j.cell.2015.05.021 (2015).
43 Graubert, T. A., DiPersio, J. F., Russell, J. H. & Ley, T. J. Perforin/granzyme-dependent and independent mechanisms are both important for the development of graft-versus-host disease after murine bone marrow transplantation. J Clin Invest 100, 904-911, doi:10.1172/jci119606 (1997).
44 Blazar, B. R., Taylor, P. A. & Vallera, D. A. CD4+and CD8+T cells each can utilize a perforin-dependent pathway to mediate lethal graft-versus-host disease in major histocompatibility complex-disparate recipients. Transplantation 64, 571-576, doi:10.1097/00007890-199708270-00004 (1997).
45 Jiang, Z., Podack, E. & Levy, R. B. Major histocompatibility complex-mismatched allogeneic bone marrow transplantation using perforin and/or Fas ligand double-defective CD4(+) donor T cells: involvement of cytotoxic function by donor lymphocytes prior to graft-versus-host disease pathogenesis. Blood 98, 390-397, doi:10.1182/blood.v98.2.390 (2001).
46 Gopalakrishnan, V. et al. Gut microbiome modulates response to anti-PD-1 immunotherapy in melanoma patients. Science 359, 97-103, doi:10.1126/science.aan4236 (2018).
47 Matson, V. et al. The commensal microbiome is associated with anti-PD-1 efficacy in metastatic melanoma patients. Science 359, 104-108, doi:10.1126/science.aao3290 (2018).
48 Routy, B. et al. Gut microbiome influences efficacy of PD-1-based immunotherapy against epithelial tumors. Science 359, 91-97, doi:10.1126/science.aan3706 (2018).
49 Chung, K. Y. et al. Phase II study of the anti-cytotoxic T-lymphocyte-associated antigen 4 monoclonal antibody, tremelimumab, in patients with refractory metastatic colorectal cancer. J Clin Oncol 28, 3485-3490, doi:10.1200/jco.2010.28.3994 (2010).
50 Le, D. T. et al. PD-1 Blockade in Tumors with Mismatch-Repair Deficiency. N Engl J Med 372, 2509-2520, doi:10.1056/NEJMoa1500596 (2015).
51 Yardeni, T. et al. Host mitochondria influence gut microbiome diversity: A role for ROS. Sci Signal 12, doi:10.1126/scisignal.aaw3159 (2019).
52 Eriguchi, Y. et al. Graft-versus-host disease disrupts intestinal microbial ecology by inhibiting Paneth cell production of alpha-defensins. Blood 120, 223-231, doi:10.1182/blood-2011-12-401166 (2012).
53 Jenq, R. R. et al. Intestinal Blautia Is Associated with Reduced Death from Graft-versus-Host Disease. Biol Blood Marrow Transplant 21, 1373-1383, doi:10.1016/j.bbmt.2015.04.016 (2015).
54 Jenq, R. R. et al. Regulation of intestinal inflammation by microbiota following allogeneic bone marrow transplantation. J Exp Med 209, 903-911, doi:10.1084/jem.20112408 (2012).
55 Shono, Y. et al. Increased GVHD-related mortality with broad-spectrum antibiotic use after allogeneic hematopoietic stem cell transplantation in human patients and mice. Sci Transl Med 8, 339ra371, doi:10.1126/scitranslmed.aaf2311 (2016).
56 Peled, J. U. et al. Intestinal Microbiota and Relapse After Hematopoietic-Cell Transplantation. J Clin Oncol 35, 1650-1659, doi:10.1200/jco.2016.70.3348 (2017).
57 Atarashi, K. et al. Induction of colonic regulatory T cells by indigenous Clostridium species. Science 331, 337-341, doi:10.1126/science.1198469 (2011).
58 Atarashi, K. et al. Treg induction by a rationally selected mixture of Clostridia strains from the human microbiota. Nature 500, 232-236, doi:10.1038/nature12331 (2013).
59 Furusawa, Y. et al. Commensal microbe-derived butyrate induces the differentiation of colonic regulatory T cells. Nature 504, 446-450, doi:10.1038/nature12721 (2013).
60 Fujiwara, H. et al. Microbial metabolite sensor GPR43 controls severity of experimental GVHD. Nat Commun 9, 3674, doi:10.1038/s41467-018-06048-w (2018).
61 Kakihana, K. et al. Fecal microbiota transplantation for patients with steroid-resistant acute graft-versus-host disease of the gut. Blood 128, 2083-2088, doi:10.1182/blood-2016-05-717652 (2016).
62 DeFilipp, Z. et al. Third-party fecal microbiota transplantation following allo-HCT reconstitutes microbiome diversity. Blood Adv 2, 745-753, doi:10.1182/bloodadvances.2018017731 (2018).
63 Biton, M. et al. T Helper Cell Cytokines Modulate Intestinal Stem Cell Renewal and Differentiation. Cell 175, 1307-1320.e1322, doi:10.1016/j.ce11.2018.10.008 (2018).
64 Cooke, K. R. et al. Tumor necrosis factor- alpha production to lipopolysaccharide stimulation by donor cells predicts the severity of experimental acute graft-versus-host disease. J Clin Invest 102, 1882-1891, doi:10.1172/jci4285 (1998).
65 Hill, G. R. et al. Interleukin-11 promotes T cell polarization and prevents acute graft-versus-host disease after allogeneic bone marrow transplantation. J Clin Invest 102, 115-123, doi:10.1172/jci3132 (1998).
66 Sas, K. M. et al. Tissue-specific metabolic reprogramming drives nutrient flux in diabetic complications. JCI Insight 1, e86976, doi:10.1172/jci.insight.86976 (2016).
67 Mathew, A. V. et al. Impaired Amino Acid and TCA Metabolism and Cardiovascular Autonomic Neuropathy Progression in Type 1 Diabetes. Diabetes 68, 2035-2044, doi:10.2337/db19-0145 (2019).
68 Mathew, A. V., Seymour, E. M., Byun, J., Pennathur, S. & Hummel, S. L. Altered Metabolic Profile With Sodium-Restricted Dietary Approaches to Stop Hypertension Diet in Hypertensive Heart Failure With Preserved Ejection Fraction. J Card Fail 21, 963-967, doi:10.1016/j.cardfail.2015.10.003 (2015).
69 Halbrook, C. J. et al. Macrophage-Released Pyrimidines Inhibit Gemcitabine Therapy in Pancreatic Cancer. Cell Metab 29, 1390-1399.e1396, doi:10.1016/j.cmet.2019.02.001 (2019).
70 Martin, S. J. et al. The cytotoxic cell protease granzyme B initiates apoptosis in a cell-free system by proteolytic processing and activation of the ICE/CED-3 family protease, CPP32, via a novel two-step mechanism. Embo j 15, 2407-2416 (1996).
71 West, A. P. et al. Mitochondrial DNA stress primes the antiviral innate immune response. Nature 520, 553-557, doi:10.1038/nature14156 (2015).
72 Mi, H. et al. Protocol Update for large-scale genome and gene function analysis with the PANTHER classification system (v.14.0). Nat Protoc 14, 703-721, doi:10.1038/s41596-019-0128-8 (2019).

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method for treating, ameliorating, or preventing a T cell mediated intestinal disorder in a patient comprising administering to a patient suffering from or at risk of suffering from a T cell mediated intestinal disorder a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

2. The method of claim 1, wherein the T cell mediated intestinal disorder is an alloimmune disorder, autoimmune disorder, and/or an iatrogenic disorder.

3. The method of claim 2, wherein the alloimmune disorder, autoimmune disorder, and/or an iatrogenic disorder selected from graft-versus-host disease (GVHD), inflammatory bowel disease (IBD) and immune checkpoint blockade (ICB) mediated colitis.

4. The method of claim 1, wherein the patient is a human patient suffering from or at risk of suffering from an alloimmune disorder, an autoimmune disorder, and/or an iatrogenic disorder.

5. The method of claim 1, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

6. A method for treating, ameliorating, or preventing a reduction of IEC related SDHA activity and/or expression in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

7. The method of claim 6, wherein the patient is a human patient suffering from or at risk of suffering from an alloimmune disorder, an autoimmune disorder, and/or an iatrogenic disorder.

8. The method of claim 6, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

9. A method for treating, ameliorating, or preventing a reduction of IEC related oxidative phosphorylation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

10. The method of claim 9, wherein the patient is a human patient suffering from or at risk of suffering from an alloimmune disorder, an autoimmune disorder, and/or an iatrogenic disorder.

11. The method of claim 9, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

12. A method for treating, ameliorating, or preventing an increase of IEC related succinate accumulation in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

13. The method of claim 12, wherein the patient is a human patient suffering from or at risk of suffering from an alloimmune disorder, an autoimmune disorder, and/or an iatrogenic disorder.

14. The method of claim 12, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

15. A method for treating, ameliorating, or preventing an increase of IEC related accumulation of perforin dependent granzyme B related to cytotoxic T cell engagement with such IECs in a patient comprising administering to a patient a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

16. The method of claim 15, wherein the patient is a human patient suffering from or at risk of suffering from an alloimmune disorder, an autoimmune disorder, and/or an iatrogenic disorder.

17. The method of claim 15, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

18. A method for reducing or preventing a reduction of IEC related SDHA activity and/or expression in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

19. The method of claim 18, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

20. The method of claim 18, wherein the biological sample is from a human patient suffering from or at risk of suffering from an alloimmune disorder, an autoimmune disorder, and/or an iatrogenic disorder.

21. The method of claim 18, wherein the biological sample is an in vivo, in vitro, in situ, or ex vivo biological sample.

22. The method of claim 18, wherein the biological sample comprises IECs.

23. A method for reducing or preventing a reduction of IEC related oxidative phosphorylation in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

24. The method of claim 23, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

25. The method of claim 23, wherein the biological sample is from a human patient suffering from or at risk of suffering from an alloimmune disorder, an autoimmune disorder, and/or an iatrogenic disorder.

26. The method of claim 23, wherein the biological sample is an in vivo, in vitro, in situ, or ex vivo biological sample.

27. The method of claim 23, wherein the biological sample comprises IECs.

28. A method for reducing or preventing an increase of IEC related succinate accumulation in a biological sample comprising exposing to the biological sample a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

29. The method of claim 28, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

30. The method of claim 28, wherein the biological sample is from a human patient suffering from or at risk of suffering from an alloimmune disorder, an autoimmune disorder, and/or an iatrogenic disorder.

31. The method of claim 28, wherein the biological sample is an in vivo, in vitro, in situ, or ex vivo biological sample.

32. The method of claim 28, wherein the biological sample comprises IECs.

33. A method for reducing or preventing an increase of IEC related accumulation of perforin dependent granzyme B related to cytotoxic T cell engagement with such IECs in a biological sample comprising exposing to the biological sample a therapeutically effective amount of a composition comprising a therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs.

34. The method of claim 33, wherein the therapeutic agent capable of preventing and/or hindering one or more of reduced IEC related SDHA activity and/or expression; reduced IEC related oxidative phosphorylation; increased IEC related succinate accumulation; and increased IEC related accumulation of perforin dependent granzyme B related to cytotoxic T cell engagement with such IECs is selected from butyrate (e.g., sodium-butyrate), and a compound structurally similar to butyrate.

35. The method of claim 33, wherein the biological sample is from a human patient suffering from or at risk of suffering from an alloimmune disorder, an autoimmune disorder, and/or an iatrogenic disorder.

36. The method of claim 33, wherein the biological sample is an in vivo, in vitro, in situ, or ex vivo biological sample.

37. The method of claim 33, wherein the biological sample comprises IECs.

38. A method for treating, ameliorating, or preventing a cancer characterized by resistance to cancer therapies (e.g., those cancer cells which are chemoresistant, radiation resistant, hormone resistant, and the like) comprising co-administering to a patient suffering from or at risk of suffering from such a cancer 1) a therapeutic agent capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs, and 2) an immune checkpoint inhibitor.

39. The method of claim 38, wherein the therapeutic agent capable of one or more of inhibiting and/or decreasing IEC related SDHA activity and/or expression; inhibiting and/or decreasing IEC related oxidative phosphorylation; increasing IEC related succinate accumulation; and increasing IEC related accumulation of perforin dependent granzyme B related to cytotoxic T cell engagement with such IECs is a SHDA inhibitor (e.g., carboxin, thenoyltrifluoroacetone, malonate, malate, oxaloacetate).

40. The method of claim 38, wherein the immune checkpoint inhibitor is selected from a PD-1 inhibitor, PD-L1 inhibitor, CTLA-4 inhibitor, LAG3 inhibitor, TIM3 inhibitor, cd47 inhibitor, TIGIT inhibitor, and B7-H1 inhibitor.
